SEQUENCE LISTING (16 sequences)

SEQ ID NO: 1 - DNA, 6532 bp, Synechocystis PCC6803
gctgtattgctcctttttgaggatttttccatgaccgttgccaccgatcgccaaactgtgcccccatctgcggcccatcctagtggagacaagcgttttaaggtgttagacgccaccatgaagcgcaaccaatttaatcaggatgccctcattgaaatcctgcataaagcccaggaaatttttggctacctggaagaggatgttctgctctacgtagcccgggggcttaaattacccctcagccgggtgtttggagtggcgactttttaccatcttttttcccttaaacccagtgggaaacatacctgtgtggtctgcttgggaacggcttgctacgttaaaggggcgggggatttgctgaaaaccctagatcaggaagtccatctgaaaccgggggaaacgacagaggatgacaaatgtccttggtgacggcccgttgcattggagcctgtgcattgccccagccgtggtctatgacggcaaagtgttgggcaagcagaatgacgaagcggtattggcggcgatacaaccttggttaagtaacagttaacggatattaagtatcaggtgattgcttgatcttttctagttgattttttgatttgttgttattgagcttaaaccccatggacattaaagaattaaaggaaattgccaccaaaagccgtgagaaacaaacaaaaattcgcattcgttgttgtagtgctgccggttgtctttcttctgaaggggagacggtgaaaaaaaatctcaccacggcgatcgccgcagcaggattggaagaaaaagtggaagtctgtggggtaggctgtatgaagttttgtggccggggccccctagtggcggtggatgaccggaatcaactctacgaatttgttaccccagaccaggtgggggatattgtcaaaaaattgcagaaacccgatgcagttgcagaaacaggcttaatcagtggtgatccccaccatcccttctacgccctgcaaaggaatattgctttggaaaattcaggccggattgatcccgaatccattgatgaatacatcgccctagggggctacgaacagcttcataaggttgtctatgaaatgaccccagaggaagtgatcgtggaaatgaacaaaagtggtctgcggggtcggggtgggggcggttatcccaccggcttgaaatgggccacagtggccaaaatgcccggccagcaaaaatatgtcatctgcaatgctgacgaaggcgatcccggtgctttcatggaccgcagtgtgttggaaagtgatccccatcgcatcctcgaaggtatggcgatcgccgcctatgcagtgggggctaaccatggttacatttatgtgcgggcggaataccccctagctatccaacgactgcaaaaagcgatccaacaggctaaacgttatggcctgatgggcacccaaatttttgactctcccattgatttcaagattgatatacgagtaggagccggtgcctttgtctgcggtgaagaaacagcattaattgcctcagtggaaggaaaacggggaacgccccgaccaagaccaccctatccagcccaatcgggtttgtggcaaagtcccaccctgattaacaatgtggaaacctacgccaacgttgtacccatcattcgggaagggggagattggtatggctccattggtacggaaaaaagtaaaggcaccaaggtttttgccctcacaggaaaagtggaaaacgctggtctgattgaagtgcccatgggaaccaccgtgcgacaagtggtggaggaaatggggggcggtgtacccaatggtggccaagtcaaagcagtgcaaactgggggcccttccggaggctgtatccccgccgataaattggatactcccatcgaatatgacaccctattagccctgggcaccatgatgggttccgggggcatgattgtcatggatgaaagcaccaatatggtggacgtggcccagttttatatggatttttgcaaatcggaatcctgtggcaaatgtattccctgccgagcgggcacagtgcaactttatgaccttttaacccgctttttagaaggggaagctacccaagaagacttgatcaaactagaaaatctttgccatatggttaaggaaactagcctttgtggattggggatgagtgcgcctaatccggtaataagtaccctgcgctattttcgtcatgaatatgaagaattactcaaagtctagttcggtaatttatccactcagttaacttttctgaaacaccatgaatgttttaactgctcccatcaaaagtgacacttggactgaggccacctgggaagaatttatccaagccactgaaaatcccgattatgacaaagcaaagttctactactatcaaaaccagttgagaattgaaatgtctcccgttggtaacgatcattcaagagaccattacctaattagtaacgctattagtctgtatgcaatttttaagaaaattccgctcaacggaaatgatacctgtagttatcgtaaacccggtcattgggaggtacaacctgatatttcttgccatgtgggggataatgctatggctatcccctctggaacaggtattgtcaatttaaatgattatcctcccccagatttagttatcgaaattgccaatacttccttagctgatgatcaaggaaaaaaacggctactttatgaagagttaggcgtcaaagaatattggattgtggatgtgaaggccactaaaatcatggggtttaaaatggaaaaccaagggagctaccaaattcgagaatctttagttttacctggattaaatttagctgttttggaagaggcgttgcaaaaaacacgccaaacgaatcatggagaagtcatgcgttggctacttcaacaatttagttaattcattttcaaaggagtttttggccaatgtctgttgttactttaaccattgatgataaggcgatcgccattgaagaaggcgcaagtattttgcaagcggctaaagaagcaggggttcccattcccaccctttgccatttagaagggatttcagaagcggcagcctgtcgtttgtgcatggtggaagtggaaggcacgaataaattgatgcccgcctgcgttaccgctgtgagcgaagaaatggtagtccacaccaacacagaaaaattgcaaaattaccgacgtatgacagtggaattacttttttccgaaggcaatcatgtctgtgccatttgtgtggctaacggcaactgtgaattgcaagatatggccattacggtgggtatggatcacagccgatttaaatatcaatttcccaagcgagaagtggatttatcccatcccatgtttggcattgatcataaccgttgtattctctgtacccgttgtgtgcgagtttgcgatgaaattgagggagcccacgtttgggatgtggcttaccggggcgcagaatgcaaaattgtttctggtttaaatcagccctggggaaccgttgatgcctgtacttcctgtggcaaatgtgtggatgcctgtcccacgggttctatcttccataaaggagaaactactgctgaaaaaattggcgatcgccgtaaggtggagtttttagccactgcccgtaaagaaaaggaatgggtcaggtaggttgaacttttaagaacttttaacatcatttctaaacttttaatcatggctaaaattcgttttgctaccgtttggctcgctggttgttccggctgtcatatgtccttccttgatatggacgaatggctcattgatctcgctcaaaaagttgatgtggttttcagtcccgttggttctgatctcaaggaatacccggacaatgtggatgtttgcctagtggaaggggcgatcgccaacgaagaaaatttagagttagctttggagttgagacagaaaacgaaggtagtaatttcctttggggactgtgctgtaaccgccaatgtccccggtatgcgtaatatgctcaaaggtagcgatccggttctgcgccgagcctatattgaactgggagatgggacgcctcaactgcccgatgaacctggtattgtgccgcctctattagacaaggttattcccctacatgaggttattccggtggatatttttatgcccggttgtcctcccgatgcccaccgtattcgagcaacgctagaaccattattaaatggggaacatcccctcatggaagggcgagcaatgatcaaatttggttaaaattcaagttttctaaacagtttgcaaaatagctattcaggagatttaataatgaatacccaattagtagaatccttggttcaaataattcaaagtctttccccagaggagcaaaagttattggaaactcatttggcagaaaaaaatagcaactggcaggaggttttggggaaaattgaaaccaatcgccaagaaatttatgcttctcgtcagggaaaaccttttgatctttctatagatgaaatcatcgaagaaatgcgtgaggaaagaacccaagatgttctacaagcctgttttggaaaatgatttttaggtatgaccaaccaaacttctttcacaatttgtattgactcaaattttattgtccgacttcttgttgggtattatgaagaaactatctatcttgagatgtggaataaatggtgtaacgcaaatactaaaattgttgctcctgatctaatcaactatgaggtgactaatgttttgtggcgtttaaacaagaccaatcagattaactacactcaagcccaaattgctcttacagaaagttttaatctcggcattgaactttattcaaactcagaactacaccaggatgctttggcgatcgccgaaaagtttcaattgtcagccgcctatgatgtccattatttagctttagcagaaaaaatgcagatagatttttatacctgtgacaaaaaactgttcaattccgtacaacaaaatttccctagaataaaattagttattgctaacagtagttagattgatttaaattcctgaatatttattacaagatccggctttctatatttactgctcaaaaaatatctaaatcaacaataatcaatcccatgtctaaaaccattgttatcgatcccgttacccggattgaaggccatgccaaaatctccattttcctcaacgaccagggcaacgtagatgatgttcgtttccatgtggtggagtatcggggttttgaaaaattttgcgaaggtcgtcccatgtgggaaatggctggtattaccgcccgtatttgcggcatttgtccggttagccatctgctctgtgcggctaaaaccggggataagttactggcggtgcaaatccctccagccggggaaaaactgcgccgtttaatgaatttagggcaaattacccaatcccacgccctaagttttttccatctcagcagtcctgattttctgcttggttgggacagtgatcccgctactcgcaatgtgtttggtttaattgctgctgaccccgatttagctagggcaggtattcggttacggcaatttggccaaacggtaattgaacttttgggagctaaaaaaatccactctgcttggtcagtgcccggtggagtccgatcgccgttgtcggaagaaggcagacaatggattgtggaccgtttaccagaagcaaaagaaaccgtttatttagccttaaatttgtttaaaaatatgttggaccgcttccaaacagaagtggcagaatttggcaaatttccctccctatttatgggcttagttgggaaaaataatgaatgggaacattatggcggctccctgcggtttaccgacagtgaaggcaatattgtcgcggacaatctcagtgaagataattacgctgattttattggtgaatcggtggaaaaatggtcctatttaaaatttccctactacaaatctctgggttatcccgatggcatttatcgggttggtccccttgcccgccttaatgtttgtcatcacattggcaccccggaagcagaccaagaattagaagaatatcggcaacgggctggaggtgtggccacgtcctctttcttttatcattacgcccgcttggtggaaattcttgcctgtttagaagccatcgaattgttaatggctgaccctgatattttgtccaaaaattgtcgagctaaggcagaaattaattgtaccgaagcggtgggagtgagcgaagcaccccggggtactttattccaccattacaagatagatgaagatggtctaattaagaaagtgaatttgatcattgccacgggcaacaataacttagccatgaataaaacagtggcccaaattgccaaacactacattcgcaatcatgatgtgcaagaagggtttttaaaccgggtggaagcgggtattcgttgttatgatccctgccttagttgttctacccatgcagcgggacaaatgccattgatgatcgatttagttaaccctcagggggaactaattaagtccatccagcgggattaaacaaaaaac

SEQ ID NO: 2 - DNA, 399 bp, Synechocystis PCC6803
atgaccgttgccaccgatcgccaaactgtgcccccatctgcggcccatcctagtggagacaagcgttttaaggtgttagacgccaccatgaagcgcaaccaatttaatcaggatgccctcattgaaatcctgcataaagcccaggaaatttttggctacctggaagaggatgttctgctctacgtagcccgggggcttaaattacccctcagccgggtgtttggagtggcgactttttaccatcttttttcccttaaacccagtgggaaacatacctgtgtggtctgcttgggaacggcttgctacgttaaaggggcgggggatttgctgaaaaccctagatcaggaagtccatctgaaaccgggggaaacgacagaggatgacaaatgtccttggtga

SEQ ID NO: 3 - protein, 133 aa, Synechocystis PCC6803
  X (133)..(133): X can be any amino acid
MTVATDRQTVPPSAAHPSGDKRFKVLDATMKRNQFNQDALIEILHKAQEIFGYLEEDVLLYVARGLKLPLSRVFGVATFYHLFSLKPSGKHTCVVCLGTACYVKGAGDLLKTLDQEVHLKPGETTEDDKCPWX

SEQ ID NO: 4 - DNA, 1602 bp, Synechocystis PCC6803
atggacattaaagaattaaaggaaattgccaccaaaagccgtgagaaacaaacaaaaattcgcattcgttgttgtagtgctgccggttgtctttcttctgaaggggagacggtgaaaaaaaatctcaccacggcgatcgccgcagcaggattggaagaaaaagtggaagtctgtggggtaggctgtatgaagttttgtggccggggccccctagtggcggtggatgaccggaatcaactctacgaatttgttaccccagaccaggtgggggatattgtcaaaaaattgcagaaacccgatgcagttgcagaaacaggcttaatcagtggtgatccccaccatcccttctacgccctgcaaaggaatattgctttggaaaattcaggccggattgatcccgaatccattgatgaatacatcgccctagggggctacgaacagcttcataaggttgtctatgaaatgaccccagaggaagtgatcgtggaaatgaacaaaagtggtctgcggggtcggggtgggggcggttatcccaccggcttgaaatgggccacagtggccaaaatgcccggccagcaaaaatatgtcatctgcaatgctgacgaaggcgatcccggtgctttcatggaccgcagtgtgttggaaagtgatccccatcgcatcctcgaaggtatggcgatcgccgcctatgcagtgggggctaaccatggttacatttatgtgcgggcggaataccccctagctatccaacgactgcaaaaagcgatccaacaggctaaacgttatggcctgatgggcacccaaatttttgactctcccattgatttcaagattgatatacgagtaggagccggtgcctttgtctgcggtgaagaaacagcattaattgcctcagtggaaggaaaacggggaacgccccgaccaagaccaccctatccagcccaatcgggtttgtggcaaagtcccaccctgattaacaatgtggaaacctacgccaacgttgtacccatcattcgggaagggggagattggtatggctccattggtacggaaaaaagtaaaggcaccaaggtttttgccctcacaggaaaagtggaaaacgctggtctgattgaagtgcccatgggaaccaccgtgcgacaagtggtggaggaaatggggggcggtgtacccaatggtggccaagtcaaagcagtgcaaactgggggcccttccggaggctgtatccccgccgataaattggatactcccatcgaatatgacaccctattagccctgggcaccatgatgggttccgggggcatgattgtcatggatgaaagcaccaatatggtggacgtggcccagttttatatggatttttgcaaatcggaatcctgtggcaaatgtattccctgccgagcgggcacagtgcaactttatgaccttttaacccgctttttagaaggggaagctacccaagaagacttgatcaaactagaaaatctttgccatatggttaaggaaactagcctttgtggattggggatgagtgcgcctaatccggtaataagtaccctgcgctattttcgtcatgaatatgaagaattactcaaagtctag

SEQ ID NO: 5 - protein, 533 aa, Synechocystis PCC6803
MDIKELKEIATKSREKQTKIRIRCCSAAGCLSSEGETVKKNLTTAIAAAGLEEKVEVCGVGCMKFCGRGPLVAVDDRNQLYEFVTPDQVGDIVKKLQKPDAVAETGLISGDPHHPFYALQRNIALENSGRIDPESIDEYIALGGYEQLHKVVYEMTPEEVIVEMNKSGLRGRGGGGYPTGLKWATVAKMPGQQKYVICNADEGDPGAFMDRSVLESDPHRILEGMAIAAYAVGANHGYIYVRAEYPLAIQRLQKAIQQAKRYGLMGTQIFDSPIDFKIDIRVGAGAFVCGEETALIASVEGKRGTPRPRPPYPAQSGLWQSPTLINNVETYANVVPIIREGGDWYGSIGTEKSKGTKVFALTGKVENAGLIEVPMGTTVRQVVEEMGGGVPNGGQVKAVQTGGPSGGCIPADKLDTPIEYDTLLALGTMMGSGGMIVMDESTNMVDVAQFYMDFCKSESCGKCIPCRAGTVQLYDLLTRFLEGEATQEDLIKLENLCHMVKETSLCGLGMSAPNPVISTLRYFRHEYEELLKV

SEQ ID NO: 6 - DNA, 639 bp, Synechocystis PCC6803
atgaatgttttaactgctcccatcaaaagtgacacttggactgaggccacctgggaagaatttatccaagccactgaaaatcccgattatgacaaagcaaagttctactactatcaaaaccagttgagaattgaaatgtctcccgttggtaacgatcattcaagagaccattacctaattagtaacgctattagtctgtatgcaatttttaagaaaattccgctcaacggaaatgatacctgtagttatcgtaaacccggtcattgggaggtacaacctgatatttcttgccatgtgggggataatgctatggctatcccctctggaacaggtattgtcaatttaaatgattatcctcccccagatttagttatcgaaattgccaatacttccttagctgatgatcaaggaaaaaaacggctactttatgaagagttaggcgtcaaagaatattggattgtggatgtgaaggccactaaaatcatggggtttaaaatggaaaaccaagggagctaccaaattcgagaatctttagttttacctggattaaatttagctgttttggaagaggcgttgcaaaaaacacgccaaacgaatcatggagaagtcatgcgttggctacttcaacaatttagttaa

SEQ ID NO: 7 - DNA, 717 bp, Synechocystis PCC6803
atgtctgttgttactttaaccattgatgataaggcgatcgccattgaagaaggcgcaagtattttgcaagcggctaaagaagcaggggttcccattcccaccctttgccatttagaagggatttcagaagcggcagcctgtcgtttgtgcatggtggaagtggaaggcacgaataaattgatgcccgcctgcgttaccgctgtgagcgaagaaatggtagtccacaccaacacagaaaaattgcaaaattaccgacgtatgacagtggaattacttttttccgaaggcaatcatgtctgtgccatttgtgtggctaacggcaactgtgaattgcaagatatggccattacggtgggtatggatcacagccgatttaaatatcaatttcccaagcgagaagtggatttatcccatcccatgtttggcattgatcataaccgttgtattctctgtacccgttgtgtgcgagtttgcgatgaaattgagggagcccacgtttgggatgtggcttaccggggcgcagaatgcaaaattgtttctggtttaaatcagccctggggaaccgttgatgcctgtacttcctgtggcaaatgtgtggatgcctgtcccacgggttctatcttccataaaggagaaactactgctgaaaaaattggcgatcgccgtaaggtggagtttttagccactgcccgtaaagaaaaggaatgggtcaggtag

SEQ ID NO: 8 - protein, 238 aa, Synechocystis PCC6803
MSVVTLTIDDKAIAIEEGASILQAAKEAGVPIPTLCHLEGISEAAACRLCMVEVEGTNKLMPACVTAVSEEMVVHTNTEKLQNYRRMTVELLFSEGNHVCAICVANGNCELQDMAITVGMDHSRFKYQFPKREVDLSHPMFGIDHNRCILCTRCVRVCDEIEGAHVWDVAYRGAECKIVSGLNQPWGTVDACTSCGKCVDACPTGSIFHKGETTAEKIGDRRKVEFLATARKEKEWVR

SEQ ID NO: 9 - DNA, 549 bp, Synechocystis PCC6803
atggctaaaattcgttttgctaccgtttggctcgctggttgttccggctgtcatatgtccttccttgatatggacgaatggctcattgatctcgctcaaaaagttgatgtggttttcagtcccgttggttctgatctcaaggaatacccggacaatgtggatgtttgcctagtggaaggggcgatcgccaacgaagaaaatttagagttagctttggagttgagacagaaaacgaaggtagtaatttcctttggggactgtgctgtaaccgccaatgtccccggtatgcgtaatatgctcaaaggtagcgatccggttctgcgccgagcctatattgaactgggagatgggacgcctcaactgcccgatgaacctggtattgtgccgcctctattagacaaggttattcccctacatgaggttattccggtggatatttttatgcccggttgtcctcccgatgcccaccgtattcgagcaacgctagaaccattattaaatggggaacatcccctcatggaagggcgagcaatgatcaaatttggttaa

SEQ ID NO: 10 - protein, 182 aa, Synechocystis PCC6803
MAKIRFATVWLAGCSGCHMSFLDMDEWLIDLAQKVDVVFSPVGSDLKEYPDNVDVCLVEGAIANEENLELALELRQKTKVVISFGDCAVTANVPGMRNMLKGSDPVLRRAYIELGDGTPQLPDEPGIVPPLLDKVIPLHEVIPVDIFMPGCPPDAHRIRATLEPLLNGEHPLMEGRAMIKFG

SEQ ID NO: 11 - DNA, 450 bp, Synechocystis PCC6803
atgaccaaccaaacttctttcacaatttgtattgactcaaattttattgtccgacttcttgttgggtattatgaagaaactatctatcttgagatgtggaataaatggtgtaacgcaaatactaaaattgttgctcctgatctaatcaactatgaggtgactaatgttttgtggcgtttaaacaagaccaatcagattaactacactcaagcccaaattgctcttacagaaagttttaatctcggcattgaactttattcaaactcagaactacaccaggatgctttggcgatcgccgaaaagtttcaattgtcagccgcctatgatgtccattatttagctttagcagaaaaaatgcagatagatttttatacctgtgacaaaaaactgttcaattccgtacaacaaaatttccctagaataaaattagttattgctaacagtagttag

SEQ ID NO: 12 - DNA, 1425 bp, Synechocystis PCC6803
atgtctaaaaccattgttatcgatcccgttacccggattgaaggccatgccaaaatctccattttcctcaacgaccagggcaacgtagatgatgttcgtttccatgtggtggagtatcggggttttgaaaaattttgcgaaggtcgtcccatgtgggaaatggctggtattaccgcccgtatttgcggcatttgtccggttagccatctgctctgtgcggctaaaaccggggataagttactggcggtgcaaatccctccagccggggaaaaactgcgccgtttaatgaatttagggcaaattacccaatcccacgccctaagttttttccatctcagcagtcctgattttctgcttggttgggacagtgatcccgctactcgcaatgtgtttggtttaattgctgctgaccccgatttagctagggcaggtattcggttacggcaatttggccaaacggtaattgaacttttgggagctaaaaaaatccactctgcttggtcagtgcccggtggagtccgatcgccgttgtcggaagaaggcagacaatggattgtggaccgtttaccagaagcaaaagaaaccgtttatttagccttaaatttgtttaaaaatatgttggaccgcttccaaacagaagtggcagaatttggcaaatttccctccctatttatgggcttagttgggaaaaataatgaatgggaacattatggcggctccctgcggtttaccgacagtgaaggcaatattgtcgcggacaatctcagtgaagataattacgctgattttattggtgaatcggtggaaaaatggtcctatttaaaatttccctactacaaatctctgggttatcccgatggcatttatcgggttggtccccttgcccgccttaatgtttgtcatcacattggcaccccggaagcagaccaagaattagaagaatatcggcaacgggctggaggtgtggccacgtcctctttcttttatcattacgcccgcttggtggaaattcttgcctgtttagaagccatcgaattgttaatggctgaccctgatattttgtccaaaaattgtcgagctaaggcagaaattaattgtaccgaagcggtgggagtgagcgaagcaccccggggtactttattccaccattacaagatagatgaagatggtctaattaagaaagtgaatttgatcattgccacgggcaacaataacttagccatgaataaaacagtggcccaaattgccaaacactacattcgcaatcatgatgtgcaagaagggtttttaaaccgggtggaagcgggtattcgttgttatgatccctgccttagttgttctacccatgcagcgggacaaatgccattgatgatcgatttagttaaccctcagggggaactaattaagtccatccagcgggattaa

SEQ ID NO: 13 - protein, 474 aa, Synechocystis PCC6803
MSKTIVIDPVTRIEGHAKISIFLNDQGNVDDVRFHVVEYRGFEKFCEGRPMWEMAGITARICGICPVSHLLCAAKTGDKLLAVQIPPAGEKLRRLMNLGQITQSHALSFFHLSSPDFLLGWDSDPATRNVFGLIAADPDLARAGIRLRQFGQTVIELLGAKKIHSAWSVPGGVRSPLSEEGRQWIVDRLPEAKETVYLALNLFKNMLDRFQTEVAEFGKFPSLFMGLVGKNNEWEHYGGSLRFTDSEGNIVADNLSEDNYADFIGESVEKWSYLKFPYYKSLGYPDGIYRVGPLARLNVCHHIGTPEADQELEEYRQRAGGVATSSFFYHYARLVEILACLEAIELLMADPDILSKNCRAKAEINCTEAVGVSEAPRGTLFHHYKIDEDGLIKKVNLIIATGNNNLAMNKTVAQIAKHYIRNHDVQEGFLNRVEAGIRCYDPCLSCSTHAAGQMPLMIDLVNPQGELIKSIQRD

SEQ ID NO: 14 - DNA, 36 bp, Artificial Sequence
  SynBamFwd primer
ccaatcatggatccgctgtattgctcctttttgagg

SEQ ID NO: 15 - DNA, 32 bp, Artificial Sequence
  SynEcoRev primer
ggattactgaattcccgtctgaatgttttttg

SEQ ID NO: 16 - DNA, 34 bp, Artificial Sequence
  SynNotRev primer
ggattactgcggccgcccgtctgaatgttttttg

(19) United States
(12) Patent Application Publication
(10) Pub. No.: US 2010/0015681 A1
(43) Pub. Date: Jan. 21, 2010

(54) Hydrogen Production By Means Of A Cell Expression System

(75) Inventors: Phillip Craig Wright, Sheffield (GB); Adam Martin Burja, Halifax (CA); Helia Radianingtyas, Halifax (CA)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The University of Sheffield, Sheffield (GB)

(21) Appl. No.: 12/097,465

(22) PCT Filed: Dec. 21, 2006
(86) PCT No.: PCT/GB2006/004832

§ 371 Date: Dec. 4, 2008

(30) Foreign Application Priority Data
Dec. 22, 2005 (GB) 0526122.7

(51) Int. Cl. C12P 3/00; C12N 15/74; C12N 1/20; C12M 1/107; C07H 21/00

(52) U.S. Cl. 435/168; 435/320.1; 435/252.3; 435/252.33; 435/300.1; 536/23.1

(57) ABSTRACT

Expression vectors, host cells and methods of using a recombinant expression system for the production of hydrogen are disclosed. The expression vectors comprise the a bidirectional hydrogenase protein complex coding sequence of SEQ ID NO:1.

The present invention relates to a recombinant expression system for the production of hydrogen by a cell. More particularly, the invention relates to an expression vector for producing a hydrogenase protein complex, derived from cyanobacteria, in a bacterial cell, typically in Escherichia coli, a host cell transformed by the expression vector, and a method for producing hydrogen by incubating the host cell under conditions suitable for photosynthetic hydrogen production.

### BACKGROUND

Hydrogen energy is a potential candidate for replacing traditional fossil fuels, in particular hydrogen produced by micro-organisms. Currently, a number of limitations exist for the photosynthetic production of hydrogen from microbial sources. Traditional hydrogen-producing micro-organisms, such as cyanobacteria and green algae, exhibit relatively low energy conversion efficiencies and low hydrogen generation rates. Additionally, there is an inherent instability in production from these organisms over time owing to various inhibitory factors. For example, the enzymes responsible are naturally oxygen-sensitive and denature in even micro-aerobic conditions.

Traditional methods have looked to advances in process control in order to increase hydrogen production from microorganisms. U.S. Pat. No. 4,532,210 discloses the production of hydrogen in an algae culture, using an alternating light/dark cycle which comprises alternating a step for cultivating the algae in water under aerobic conditions in the presence of light to accumulate photosynthetic products in the algae and a step for cultivating the algae in water under microaerobic conditions in the dark to decompose accumulated material by respiration to evolve hydrogen.

More recently, molecular techniques have been employed to address the issue. U.S. Pat. No. 6,858,718 discloses that the enzyme, iron hydrogenase (HydA), has industrial applications for the production of hydrogen, specifically, for catalyzing the reversible reduction of protons to molecular hydrogen. The document discloses the isolation of a nucleic acid sequence from the algae Scenedesmus obliquus, Chlamydomonas reinhardtii, and Chlorella fusca that encode iron hydrogenases. The invention further discloses the genomic nucleic acid, cDNA and the protein sequences for HydA. Hitherto, none of the methods proposed have been suitable for the production of hydrogen on an industrial scale.

The present disclosure relates to the expression of an enzyme or enzyme complex isolated from a photosynthetic bacterial species, for example a cyanobacterial species, in a host cell, typically a bacterial host cell that does not express said enzyme or enzyme complex; and the production of hydrogen by said host cell.

### BRIEF SUMMARY OF THE DISCLOSURE

According to an aspect of the invention there is provided an expression vector for producing a hydrogenase protein or hydrogenase protein complex, comprising the operably linked elements of:
a) a transcriptional promoter element;
b) a nucleic acid molecule which encodes a polypeptide having the specific enzyme activity associated with a cyanobacterial hydrogenase; and
c) a transcriptional terminator.

Preferably, the nucleic acid molecule is selected from the group consisting of:
i) a nucleic acid molecule comprising the nucleotide sequence of SEQ ID NO: 1;
ii) a nucleic acid molecule having at least 70% identity to the nucleotide sequence of SEQ ID NO: 1;
iii) a nucleic acid molecule which hybridizes to the nucleic acid
   sequence of SEQ ID NO:1 and encodes a polypeptide with hydrogenase activity; or
iv) a nucleic acid molecule comprising a nucleotide sequence that is degenerate as a result of the genetic code to the sequences of i), ii) and iii) above.

More preferably, the nucleic acid molecule consists of the nucleotide sequence of SEQ ID NO: 1.

Alternatively, the nucleic acid molecule is selected from the group consisting of:
i) a nucleic acid molecule comprising the nucleotide sequence of each of SEQ ID NO:'s 2, 4, 7, 9 and 12;
ii) a nucleic acid molecule comprising a nucleotide sequence having at least 70% identity to SEQ ID NO:2, a nucleotide sequence having at least 70% identity to SEQ. ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11; or
iii) a nucleic acid molecule consisting of a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11.

More preferably, the nucleic acid molecule consists of the nucleotide sequence of each of SEQ ID NO:'s 2, 4, 7, 9 and 12.

Alternatively, the nucleic acid molecule is selected from the group consisting of:
i) a nucleic acid molecule comprising the nucleotide sequence of at least one of SEQ ID NO:'s 2, 4, 7, 9 or 12; or
ii) a nucleic acid molecule comprising the nucleotide sequence of at least one of a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11.

More preferably, the nucleic acid molecule is a nucleic acid molecule represented by the nucleic acid sequence in SEQ ID NO:2, or a variant nucleic acid molecule that hybridises to SEQ ID NO: 2 and encodes a polypeptide that has diaphorase activity. Alternatively, the nucleic acid molecule is a nucleic acid molecule represented by the nucleic acid sequence in SEQ ID NO:4, or a variant nucleic acid molecule that hybridises to SEQ ID NO: 4 and encodes a polypeptide that has NADH dehydrogenase I activity. Alternatively, the nucleic acid molecule is a nucleic acid molecule represented by the nucleic acid sequence in SEQ ID NO:7, or a variant nucleic acid molecule that hybridises to SEQ ID NO: 7 and encodes a polypeptide that has NAD reducing. hydrogenase gamma activity. Alternatively, the nucleic acid molecule is a nucleic acid molecule represented by the nucleic acid sequence in SEQ ID NO:9, or a variant nucleic acid molecule that hybridises to SEQ ID NO: 9 and encodes a polypeptide that has NAD reducing hydrogenase delta activity. Alternatively, the nucleic acid molecule is a nucleic acid molecule represented by the nucleic acid sequence in SEQ ID NO:12, or a variant nucleic acid molecule that hybridises to SEQ ID NO: 12 and encodes a polypeptide that has NAD reducing hydrogenase beta activity. Preferably, the nucleic acid molecules hybridise under stringent hybridisation conditions.

Preferably, the nucleic acid molecule consists of a nucleotide sequence that encodes each polypeptide of SEQ ID NO's: 3, 5, 8, 10 and 13.

Preferably, the variant nucleic acid molecule hybridises under stringent hybridisation conditions.

Preferably, the transcription promoter element comprises an element that confers inducible expression on said nucleic acid molecule or variant nucleic acid molecule. Alternatively, the promoter element comprises an element that confers repressible expression on said nucleic acid molecule or variant nucleic acid molecule. Alternatively, the transcription promoter element confers constitutive expression on said nucleic acid molecule or variant nucleic acid molecule.

Preferably, the expression vector includes a selectable marker. Preferably, the expression vector comprises a translational control element. Preferably, said translational control element is a ribosomal binding sequence.

Preferably said nucleic acid molecule comprises specific changes in the nucleotide sequence so as to optimize codon usage, introduced for example by DNA shuffling, error prone PCR or site directed mutagenesis.

In a further aspect, the invention provides a host cell transformed with the expression vector according to a first aspect of the invention.

Preferably said cell is a bacterial cell, more preferably a Gram negative bacterial cell, for example of the genus Escherichia spp, preferably Escherichia coli, more preferably Escherichia coli BL21 or Escherichia coli BL21 (DE3)pLys5. Alternatively, the cell may be another bacterial cell, for example a Gram positive bacterial cell, or alternatively a yeast cell, an algae cell, an insect cell, or a plant cell.

Preferably, said cell comprises a vector comprising tRNA genes, for example tRNA genes that encode for argU, ilex, leuW, proL or glyT.

According to a further aspect of the invention there is provided a method for producing hydrogen comprising:
i) incorporating a nucleic acid molecule comprising at least one cyanobacteria hydrogenase gene into an expression vector for expression in a host cell; and
ii) transfecting a host cell with the expression vector;
   wherein the resulting transfected host cell produces hydrogen.

Preferably, said at least one hydrogenase gene is a bidirectional hydrogenase gene. Preferably, said cyanobacterium is of the genus Synechocystis, more preferably Synechocystis sp. PCC 6803.

Preferably, the nucleic acid molecule is selected from the group consisting of:
i) a nucleic acid molecule comprising the nucleotide sequence of SEQ ID NO: 1;
ii) a nucleic acid-molecule having at least 70% identity to the nucleotide sequence of SEQ ID NO: 1;
iii) a nucleic acid molecule which hybridizes to the nucleic acid sequence of SEQ ID NO:1; or
iv) a nucleic acid molecule comprising a nucleotide sequence that is degenerate as a result of the genetic code to the sequences of i), ii) and iii) above.

More preferably, the nucleic acid molecule consists of the nucleotide sequence of SEQ ID NO: 1.

Alternatively, the nucleic acid molecule is selected from the group consisting of:
i) a nucleic acid molecule comprising the nucleotide sequence of each of SEQ ID NO:'s 2, 4, 7, 9 and 12;
ii) a nucleic acid molecule comprising a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11; or
iii) a nucleic acid molecule consisting of a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11.

More preferably, the nucleic acid molecule consists of the nucleotide sequence of each of SEQ ID NO:'s 2, 4, 7, 9 and 12.

Alternatively, the nucleic acid molecule is selected from the group consisting of:
i) a nucleic acid molecule comprising the nucleotide sequence of at least one of SEQ ID NO:'s 2, 4, 7, 9 or 12; or
ii) a nucleic acid molecule comprising the nucleotide sequence of at least one of a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11.

More preferably, the nucleic acid molecule is a nucleic acid molecule represented by the nucleic acid sequence in SEQ ID NO:2, or a variant nucleic acid molecule that hybridises to SEQ ID NO: 2 and encodes a polypeptide that has diaphorase activity. Alternatively, the nucleic acid molecule is a nucleic acid molecule represented by the nucleic acid sequence in SEQ ID NO:4, or a variant nucleic acid molecule that hybridises to SEQ ID NO: 4 and encodes a polypeptide that has NADH dehydrohgenase I activity. Alternatively, the nucleic acid molecule is a nucleic acid molecule represented by the nucleic acid sequence in SEQ ID NO:7, or a variant nucleic acid molecule that hybridises to SEQ ID NO: 7 and encodes a polypeptide that has NAD reducing hydrogenase gamma activity. Alternatively, the nucleic acid molecule is a nucleic acid molecule represented by the nucleic acid sequence in SEQ ID NO:9, or a variant nucleic acid molecule that hybridises to SEQ ID NO: 9 and encodes a polypeptide that has NAD reducing hydrogenase delta activity. Alternatively, the nucleic acid molecule is a nucleic acid molecule represented by the nucleic acid sequence in SEQ ID NO:12, or a variant nucleic acid molecule that hybridises to SEQ ID NO: 12 and encodes a polypeptide that has NAD reducing hydrogenase beta activity. Preferably, the nucleic acid molecules hybridise under stringent hybridisation conditions.

Preferably, the nucleic acid molecule consists of a nucleotide sequence that encodes each polypeptide of SEQ ID NO's: 3, 5, 8, 10 and 13.

According to a further aspect the invention there is provided a reaction vessel containing a host cell according to the invention and medium sufficient to support the growth of said cell. In a preferred embodiment the vessel is a bioreactor, for example a fermentor.

In a further aspect the invention there is provided a method for producing hydrogen comprising:
i) providing a vessel containing a host cell according to the invention;
ii) providing cell culture conditions which facilitate hydrogen production by a cell culture contained in the vessel; and optionally
iii) collecting hydrogen from the vessel.

According to a further aspect of the invention there is provided an apparatus for the production and collection of hydrogen by a cell comprising:
i) a reaction vessel containing a host cell according to the invention ; and
ii) a second vessel in fluid connection with said cell culture vessel wherein said second vessel is adapted for the collection and/or storage of hydrogen produced by cells contained in the cell culture vessel in (i).

According to a further aspect of the invention there is provided the use of a cyanobacterial hydrogenase in a recombinant expression system for the production of hydrogen. Preferably, the cyanobacterial hydrogenase is encoded by a nucleic acid molecule selected from the group consisting of:
i) a nucleic acid molecule comprising the nucleotide sequence of SEQ ID NO: 1;
ii) a nucleic acid molecule having at least 70% identity to the nucleotide sequence of SEQ ID NO: 1 and which encodes a polypeptide that has hydrogenase activity;
iii) a nucleic acid molecule which hybridizes to the nucleic acid sequence of SEQ ID NO:1 and which encodes a polypeptide that has hydrogenase activity; or
iv) a nucleic acid molecule comprising a nucleotide sequence that is degenerate as a result of the genetic code to the sequences of i), ii) and iii) above.

According to a further aspect of the invention there is provided a nucleic acid molecule represented by the nucleic acid sequence in SEQ ID NO:1.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction. with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE DRAWINGS

FIG. 1 is a 1:1000 scaled schematic illustration of all hydrogen metabolism associated genes within the entire Synechocystis sp. PCC 6803 genome;

FIG. 2 is a schematic illustration of the hox operon within the Synechocystis sp. PCC 6803 genome;

FIG. 3 is a schematic illustration of expression vector pET-17b;

FIG. 4 is a schematic representation of the expression vector of the invention comprising a nucleic acid molecule having the nucleotide sequence of SEQ ID NO:1;

FIG. 5 is the nucleotide sequence of SEQ ID NO:1;

FIG. 6 is the nucleotide sequence of SEQ ID NO:2;

FIG. 7 is the amino acid sequence of SEQ ID NO:3;

FIG. 8 is the nucleotide sequence of SEQ ID NO:4;

FIG. 9 is the amino acid sequence of SEQ ID NO:5;

FIG. 10 is the nucleotide sequence of SEQ ID NO:6;

FIG. 11 is the nucleotide sequence of SEQ ID NO:7;

FIG. 12 is the amino acid sequence of SEQ ID NO:8;

FIG. 13 is the nucleotide sequence of SEQ ID NO:9;

FIG. 14 is the amino acid sequence of SEQ ID NO:10;

FIG. 15 is the nucleotide sequence of SEQ ID NO:11;

FIG. 16 is the nucleotide sequence of SEQ ID NO:12; and

FIG. 17 is the amino acid sequence of SEQ ID NO:13.

### DETAILED DESCRIPTION

Microalgae (green algae and cyanobacteria) possess certain distinct advantages over higher plants when grown as solar energy harvesters; they grow at a faster rate, are easier to manipulate in open ponds or closed reactors, and generally possess a higher photosynthetic efficiency. The inherent ability of cyanobacteria and green algae to produce H₂ from water may be adapted to advantage in the development of low carbon clean energy technologies. This ability depends on the activity of up to two different hydrogenases. One is the dimeric membrane-bound hydrogenase, which is mainly confined to heterocysts and functions in reutilising the H₂-gas produced by the nitrogenase. The second is the bidirectional hydrogenase, an enzyme that can recombine and consume photosynthetically-generated electrons and protons to both evolve and degrade H₂

Synechocystis sp. PCC 6803 is a unicellular non-nitrogen-fixing cyanobacterium and an inhabitant of fresh water. This strain is naturally transformable by exogenous DNA (i.e., it takes up DNA by itself), it is spontaneously transformable, and it can integrate DNA into its genome by homologous recombination. The organism can grow under a number of different conditions, ranging from photoautotrophic to fully heterotrophic modes, making genetic modifications which interfere with basic process, such as studies of photosynthesis (and in this case hydrogenase), feasible. These properties make Synechocystis sp. PCC 6803 a favoured choice for genetic manipulations, such as those described here. In fact, this organism has been shown to lack a functioning uptake hydrogenase enzyme (due to the lack of a large subunit). This feature further increases the ‘usefulness’ of this organism within this instance, thus removing the detrimental influence of the uptake hydrogenase allowing for exacting in vivo screening of hydrogenase activity without the need to take into account the counter-productive (in this case) effects of the uptake hydrogenase.

Five genes have been described to form the bidirectional hydrogenase enzyme complex, four being homologous to genes encoding the tetrameric NAD⁺-reducing hydrogenase of Ralstonia eutrophia, where the diaphorase moiety is encoded by hoxFU and the hydrogenase part by hoxYH. In contrast to the soluble enzyme within R. eutrophia, the gene cluster of the bidirectional hydrogenase of Synechocystis sp. PCC 6803 contains a further open reading frame (hoxE), thought to encode a third diaphorase subunit. Thus, HoxEFU has been postulated to serve as the NADH oxidising part of complex I either active in respiration or cyclic electron transport around photosystem I, mainly due to significant sequence similarities to three subunits of the mitochondrial complex I (NADH:Q oxidoreductase), with HoxE being homologous to NuoE of Escherichia coli (one of the three subunit constituents the hydrophilic part of complex I). Selective isolation experiments have determined that activity is noted within unicellular and both the heterocyst and vegetative cells of heterocystous cyanobacterial species.

Cyanobacterial hydrogen production can be derived from the activity of the nitrogenase or the bidirectional hydrogenases. The net H₂ evolution by cyanobacteria is thus the sum of H₂ production catalysed by the nitrogenase and bidirectional hydrogenase and H₂ consumption catalysed by the uptake hydrogenase. The present application is concerned with the generation of hydrogen via the bidirectional hydrogenase enzyme (1), due to the significantly increased energy efficiency of this reaction compared to that of the nitrogenase (2), as illustrated below:

2H⁺+2e⁻+2NADP→H₂+2NAD⁺+2Pᵢ (1)

N₂+8H⁺+8e⁻+16ATP→2NH₃+H₂+16ADP+16Pᵢ (2)

Hydrogenase related genes which have been shown to be present within Synechocystis sp. PCC 6803 include: (1) sll0322—hydrogenase maturation protein HypF (hypF), (2) sll1078—hydrogenase expression/formation protein HypA (hypA), (3) sll1079—hydrogenase expression/formation protein HypB (hypB), (4)sll1220—NADH dehydrogenase I chain E (hoxE), (5) sll1221—NADH dehydrogenase I chain F (hoxF), (6) sll1223—NAD-reducing hydrogenase HoxS gamma subunit (hoxU), (7) sll1224—NAD-reducing hydrogenase HoxS delta subunit (hoxY) (EC. 1.12.1.2), (8) sll1226—NAD-reducing hydrogenase HoxS beta subunit (hoxH), (9) sll1432—hydrogenase isoenzymes formation (nickel incorporation) protein HypB (hypB), (10) sll1462—hydrogenase expression/formation protein HypE (hypE), (11) s111559—soluble hydrogenase 42 kD subunit, (12) slr1498—hydrogenase isoenzymes formation protein HypD (hypD), (13) slr1675—hydrogenase formation (nickel incorporation) protein HypA (hypA), (14) slr2135—hydrogenase accessory protein, (15) ssl3580—hydrogenase expression/formation protein HypC (hypC).

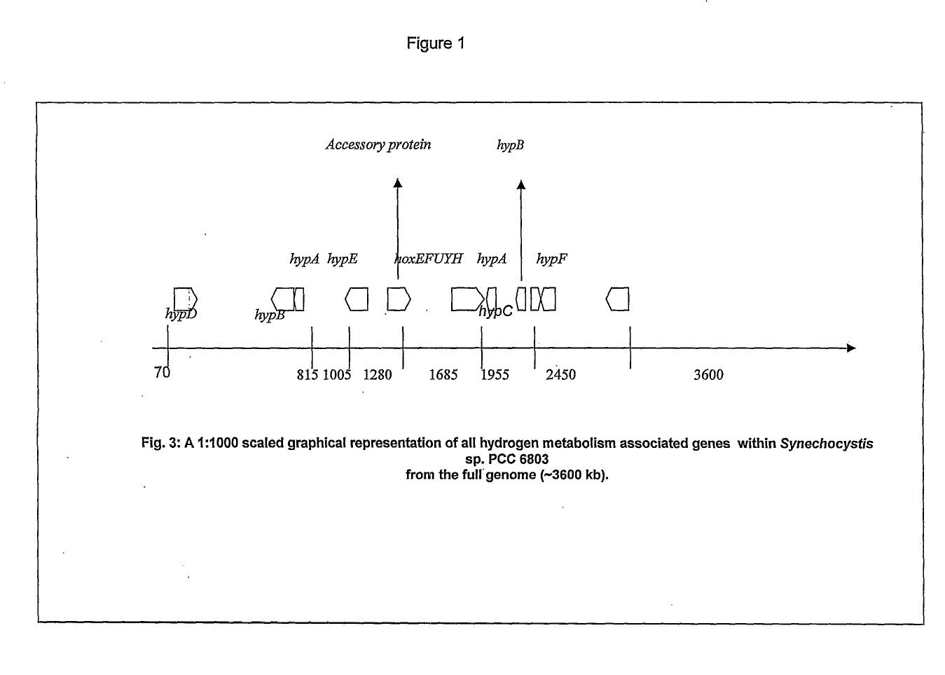

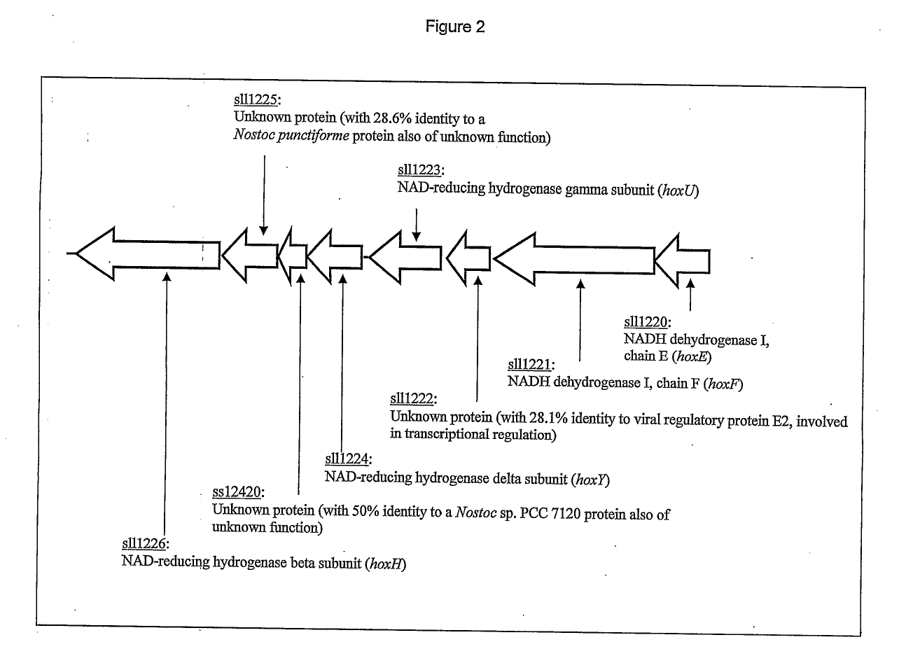

A plot of the exact location of all of these hydrogenase related genes within Synechocystis sp. PCC 6803, is illustrated in FIG. 1, a location map which covers approximately 75% of the complete genome of this organism. Therefore, the present invention utilises sequences derived from the hox operon of Synechocystis sp. PCC 6803, illustrated in FIG. 2 which is approximately 7 kb in length.

### Vector

As used herein, the term “vector” refers to a nucleic acid molecule capable of transporting another nucleic acid to which it has been linked. The vector can be capable of autonomous replication or it can integrate into a host DNA. The vector may include restriction enzyme sites for insertion of recombinant DNA and may include one or more selectable markers. The vector can be a nucleic acid in the form of a plasmid, a bacteriophage or a cosmid. Most preferably the vector is suitable for bacterial expression, e.g. for expression in E. coli, Bacillus subtilis, Salmonella, Staphylococcus, Streptococcus, Saccharomycetes, etc.

Preferably the vector is capable of propagation in the bacterial cell and is stably transmitted to future generations.

“Operably linked” as used herein, refers to a single or a combination of the above-described control elements together with a coding sequence in a functional relationship with one another, for example, in a linked relationship so as to direct expression of the coding sequence.

“Regulatory sequences” as used herein, refers to, DNA or RNA elements that are capable of controlling gene expression. Examples of expression control sequences include promoters, enhancers, silencers, Shine Dalgarno sequences, TATA-boxes, internal ribosomal entry sites (IRES), attachment sites for transcription factors, transcriptional terminators, polyadenylation sites, RNA transporting signals or sequences important for UV-light mediated gene response. Preferably the expression vector includes one or more regulatory sequences operatively linked to the nucleic acid sequence to be expressed. Regulatory sequences include those which direct constitutive expression, as well as tissue-specific regulatory and/or inducible sequences.

“Promoter”, as used herein, refers to the nucleotide sequences in DNA or RNA to which RNA polymerase binds to begin transcription. The promoter may be inducible or constitutively expressed. Alternatively, the promoter is under the control of a repressor or stimulatory protein. Preferably the promoter is a T7, T3, lac, lac UV5, tac, trc, [lambda]PL, Sp6 or a UV-inducible promoter. More preferably the promoter is a T7 or T3 promoter, known to be functional in bacteria, for example E. coli.

“Transcriptional terminator” as used herein, refers to a DNA element, which terminates the function of RNA polymerases responsible for transcribing DNA into RNA. Preferred transcriptional terminators are characterized by a run of T residues preceded by a GC rich dyad symmetrical region. More preferably transcriptional terminators are terminator sequences from the T7 phage.

“Translational control element”, as used herein, refers to DNA or RNA elements that control the translation of mRNA. Preferred translational control elements are ribosome binding sites. Preferably, the translational control element is from a homologous system as the promoter, for example a promoter and it's associated ribozyme binding site. Preferred ribosome binding sites are T7 or T3 ribosome binding sites.

“Restriction enzyme recognition site” as used herein, refers to a motif on the DNA recognized by a restriction enzyme.

“Selectable marker” as used herein, refers to proteins that, when expressed in a host cell, confer a phenotype onto the cell which allows a selection of the cell expressing said selectable marker gene. Generally this may be a protein that confers resistance to an antibiotic such as ampicillin, kanamycin, chloramphenicol, tetracyclin, hygromycin, neomycin or methotrexate. Further examples of antibiotics are Penicillins; Ampicillin HCl, Ampicillin Na, Amoxycillin Na, Carbenicillin sodium, Penicillin G, Cephalosporins, Cefotaxim Na, Cefalexin HCl, Vancomycin, Cycloserine. Other examples include Bacteriostatic Inhibitors such as: Chloramphenicol, Erythromycin, Lincomycin, Tetracyclin, Spectinomycin sulfate, Clindamycin HCl, Chlortetracycline HCl.

The design of the expression vector depends on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., the Synochocystis sp. PCC 6803 bidirectional hydrogenase protein complex, i.e., the hoxE, hoxF, hoxU, hoxY and hoxH protein subunits).

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present invention.

Preferably the vector comprises those genetic elements which are necessary for expression of the bidirectional hydrogenase protein complex in the bacterial cell. The elements required for transcription and translation in the bacterial cell include a promoter, a coding region for the bidirectional hydrogenase protein complex, and a transcriptional terminator.

Expression vectors of the invention can be bacterial expression vectors, for example recombinant bacteriophage DNA, plasmid DNA or cosmid DNA, yeast expression vectors e.g. recombinant yeast expression vectors, vectors for expression in insect cells, e.g., recombinant virus expression vectors, for example baculovirus, or vectors for expression in plant cells, e.g. recombinant virus expression vectors such as cauliflower mosaic virus, CaMV, tobacco mosaic virus, TMV, or recombinant plasmid expression vectors such as Ti plasmids.

Preferably, the vector is a bacterial expression vector. Preferably, the expression vector is a high-copy-number expression vector; alternatively, the expression vector is a low—copy-number expression vector, for example, a Mini-F plasmid.

Preferably, the vector is a bacterial expression vector comprising a T7 promoter system. Alternatively, the vector is bacterial expression vector comprising a tac promoter system.

More preferably, the vector is a pET expression vector. For example the vector can be a Novogen® pET vector, such as pET-3a, pET-3b, pET-3c, pET-3d, pET-9a, pET-9b, pET-9c, pET-9d, pET-11a, pET-11b, pET-11c, pET-11d, pET-12a, pET-12b, pET-12c, pET-14b, pET-15b, pET-16b, pET-17b, pET-17xb, pET-19b, pET-20b(+), pET-21(+), pET-21a(+), pET-21b(+), pET-21c(+), pET-21d(+), pET-22b(+), pET-23(+), pET-23a(+), pET-23b(+), pET-23c(+), pET-23d(+), pET-24(+), pET-24a(+), pET-24b(+), pET-24c(+), pET-24d(+), pET-25b(+), pET-26b(+), pET-27b(+), pET-28a(+), pET-28b(+), pET-28c(+), pET-29a(+), pET-29b(+), pET-29c(+), pET-30 Ek/LIC, pET-30 Xa/LIC, pET-30a(+), pET-30b(+), pET-30c(+), pET-31b(+), pET-32 Ek/LIC, pET-32 Xa/LIC, pET-32a(+), pET-32b(+), pET-32c(+), pET-33b(+), pET-39b(+), pET-40b(+), pET-41a(+), pET-41b(+), pET-41c(+), pET-41 Ek/LIC, pET-42a(+), pET-42b(+), pET-42c(+), pET-43.1a(+), pET-43.1b(+), pET-43.1c(+), pET-43.1 Ek/LIC, pET-44a(+), pET-44b(+), pET-44c(+), pET-44 Ek/LIC, pET-45b(+), pET-46 Ek/LIC, pET-47b(+), pET-48b(+), pET-49b(+), pET-50b(+), pLacl, pLysE, pLysS, or an Invitrogen® pET vector, for example pET161-DEST, pET101/D-TOPO pET151/D/LacZ pET104.1-DEST pET161-GW/CAT pET104.1/GW/lacZ pET SUMO/CAT pET SUMO pET-DEST41 pET-DEST42 pET101/D/LacZ pET151/D-TOPO pET161-DEST pET100/D/LacZ pET161-GW/CAT pET151/D/LacZ pET101/D-TOPO pET104-DEST pET160-DEST pET102/D/LacZ pET200/D/LacZ pET200/D-TOPO pET161/GW/D-TOPO pET160-GW/CAT.

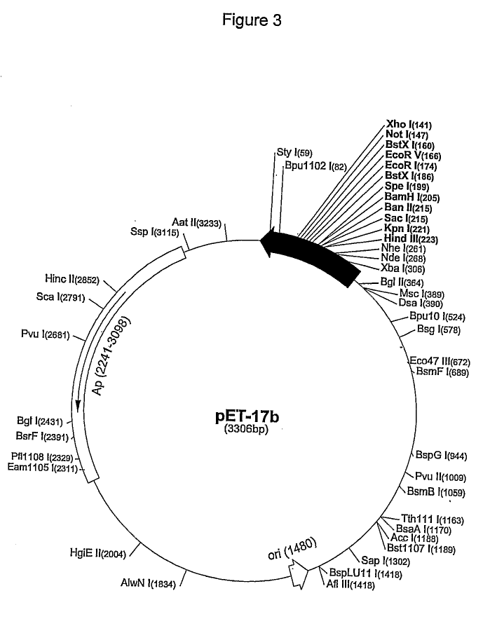

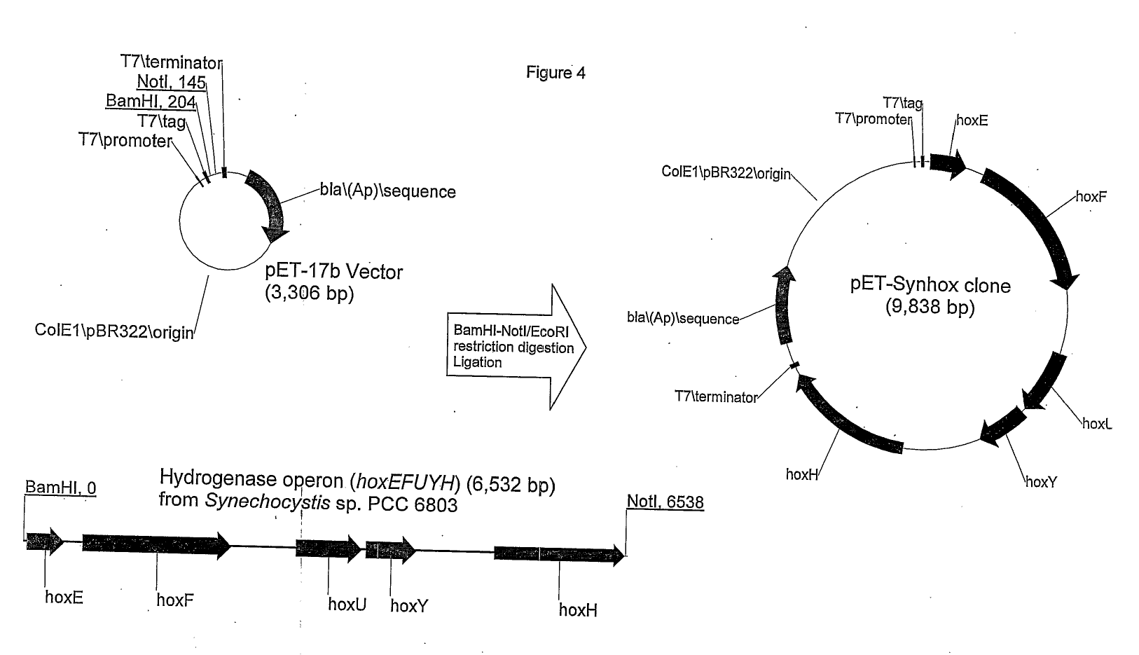

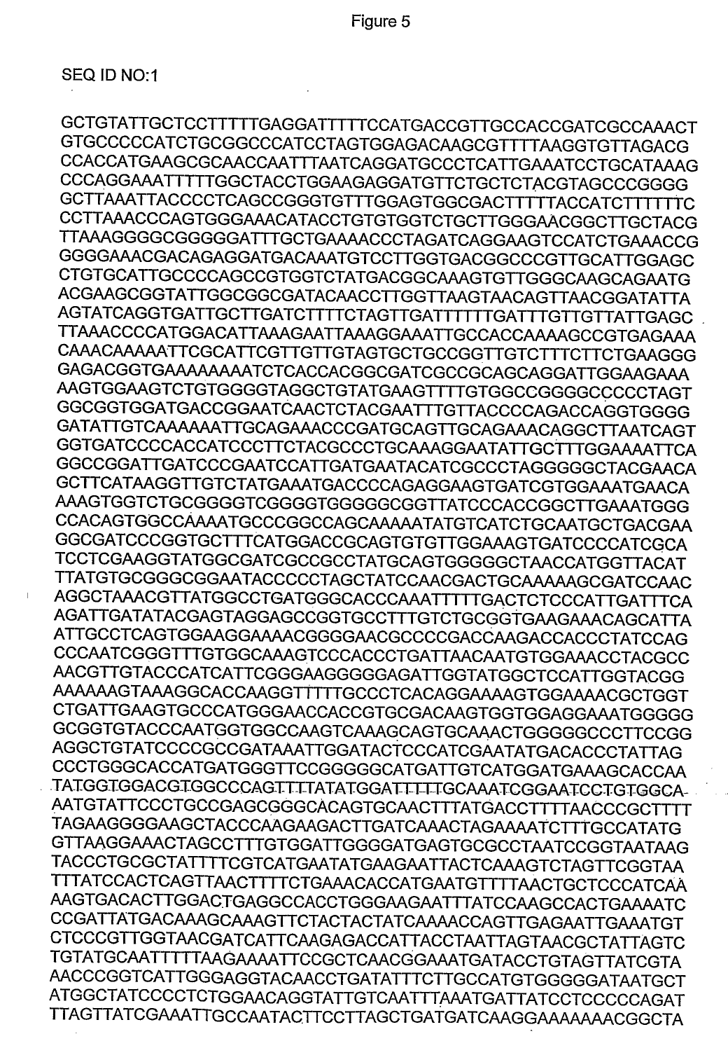

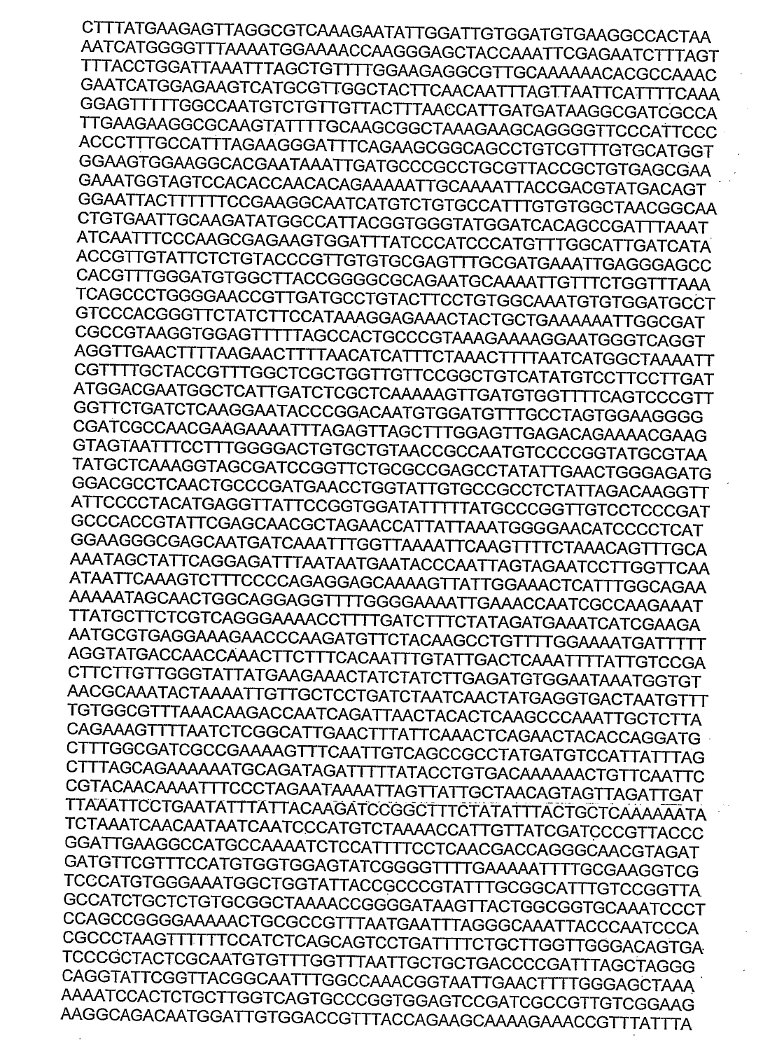

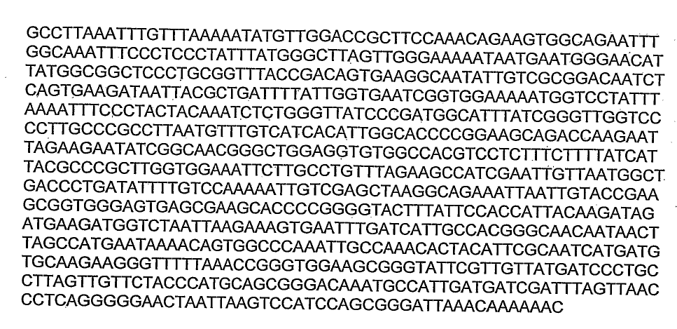

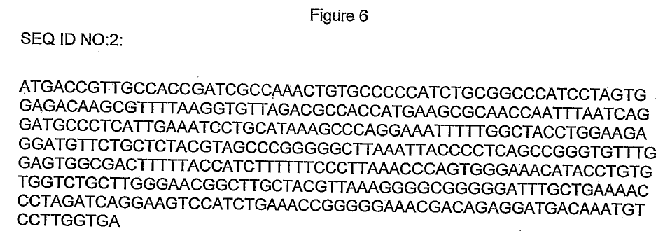

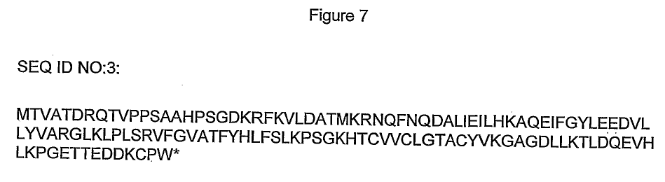

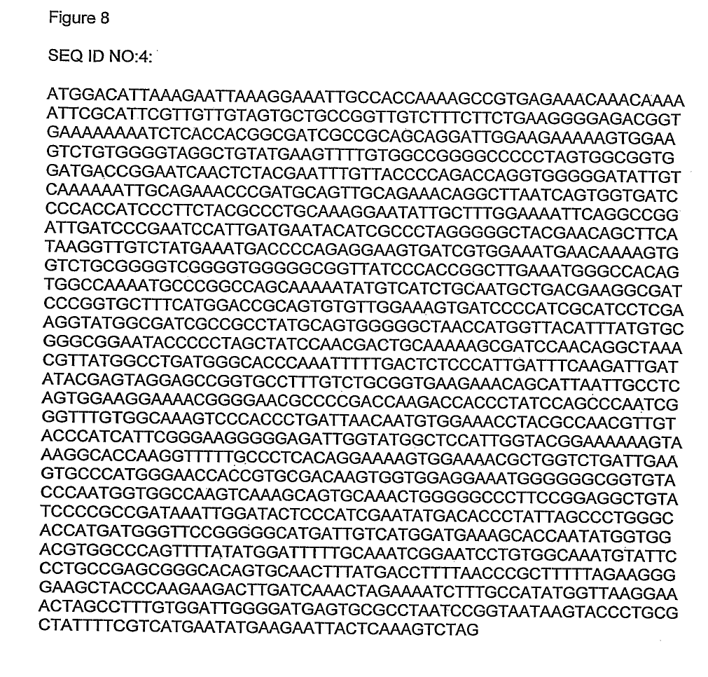

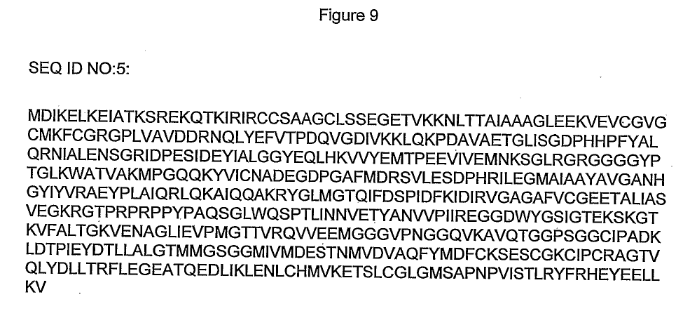

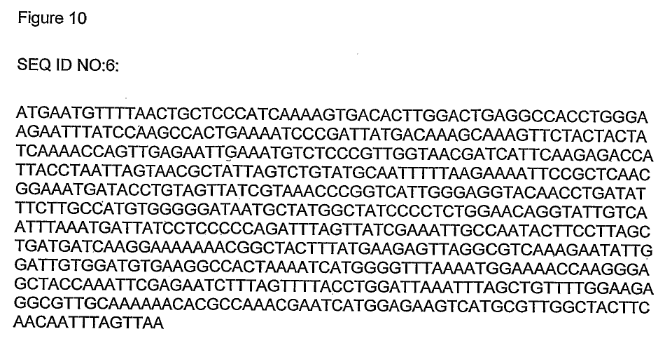

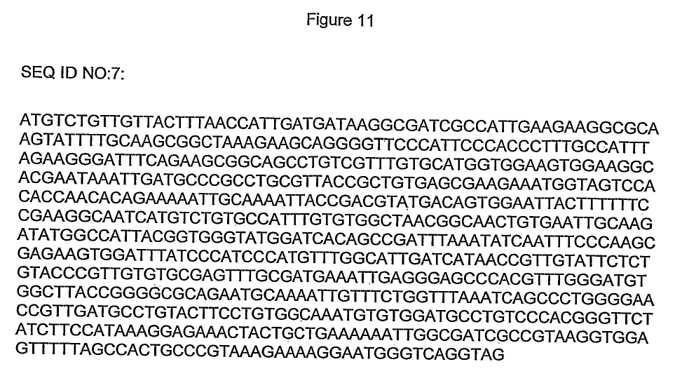

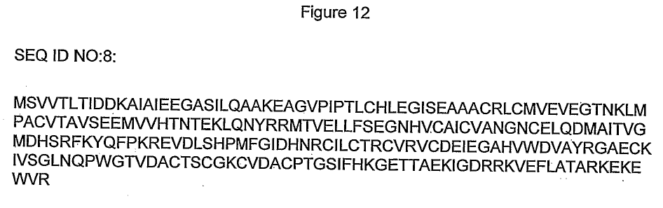

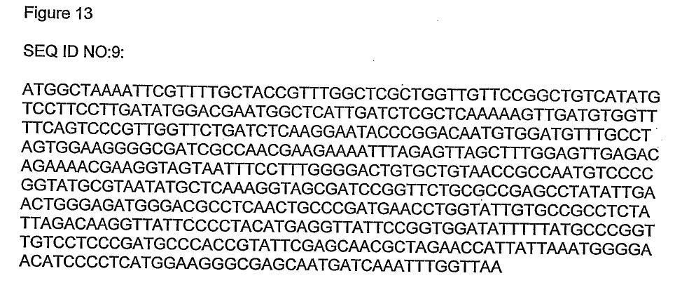

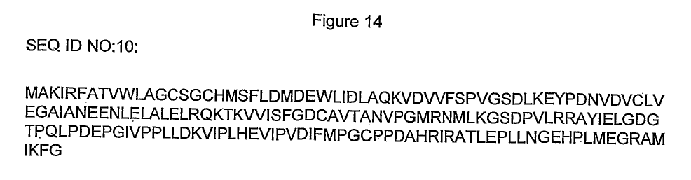

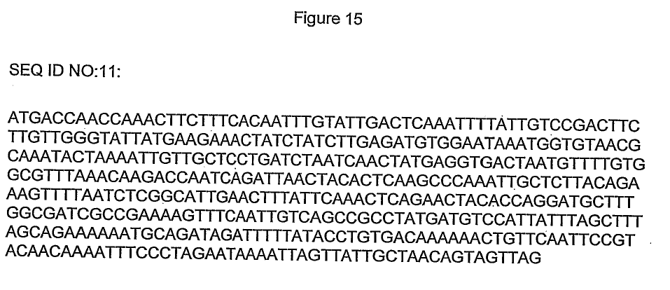

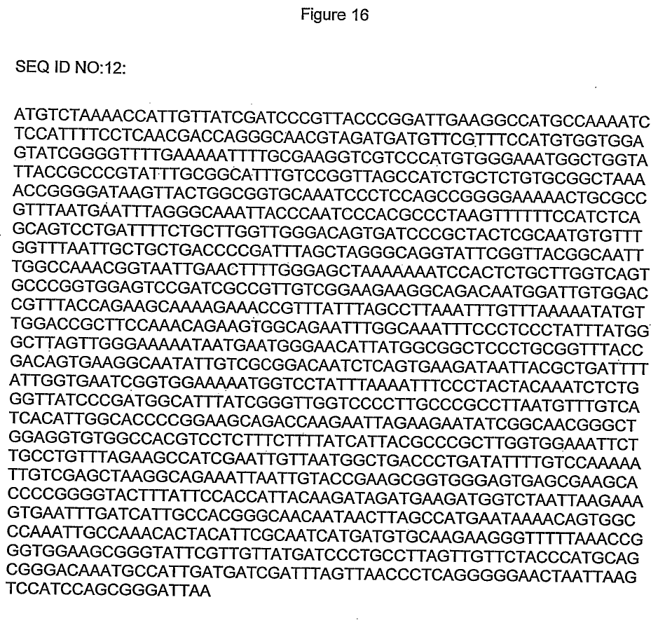

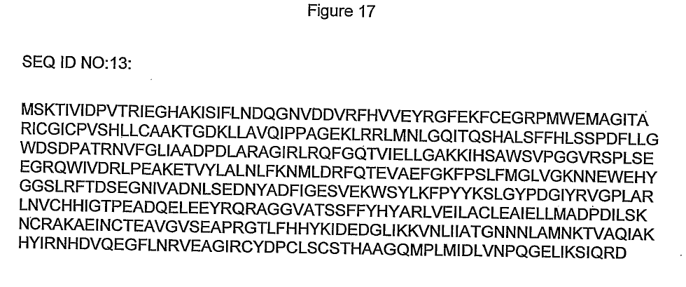

More preferably the vector is pET-17b shown in FIG. 3 (Novagen®, Madison, Wis., USA), (Seed, B. (1987) Nature 329, 840). The pET-17b vector carries an N-terminal 11 aa T7-Tag sequence followed by a region of useful cloning sites. Included in the multiple cloning regions are dual BstX I sites, which allow efficient cloning using an asymmetric linker. Unique sites are shown on the circle map of FIG. 3. The sequence is numbered by the Pbr322 convention, so the T7 expression region is reversed on the circular map. The cloning/expression region of the coding strand transcribed by T7 RNA polymerase is shown in FIG. 4.

pET-17b vector comprises a T7 promoter (nucleic acids 333-349), a T7 transcription start (nucleic acid 332) and a T7 terminator (nucleic acids 28-74). The pET-17b vector further comprises a T7-Tag sequence which allows for affinity purification of an expressed enzyme. The pET-17b vector is a translation vector which expresses from the GAT triplet following the BamHI recognition site.

In particular, the use of a vector containing the T7 promoter region, e.g. pET-17b, requires the host cell be appropriate for high protein expression.

Synechocystis sp. PCC 6803 Hox Operon

As used herein, the term “nucleic acid molecule” includes DNA molecules (e.g., a cDNA or genomic DNA) and RNA molecules (e.g., a mRNA) and analogs of the DNA or RNA generated, e.g., by the use of nucleotide analogs. The nucleic acid molecule can be single-stranded or double-stranded, but preferably is double-stranded DNA.

With regards to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′- and/or 3′-ends of the nucleic acid) in the genomic DNA of the organism from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in available references (e.g., Current Protocols in Molecular Biology, John Wiley & Sons, N.Y., 1989, 6.3.1-6.3.6). Aqueous and non-aqueous methods are described in that reference and either can be used. A preferred example of stringent hybridization conditions are hybridization in 6× sodium chloride/sodium citrate (SSC) at about 45° C., followed by one or more washes in 0.2×SSC, 0.1% (w/v) SDS at 50° C. Another example of stringent hybridization conditions are hybridization in 6×SSC at about 45° C., followed by one or more washes in 0.2×SSC, 0.1% (w/v) SDS at 55° C. A further example of stringent hybridization conditions are hybridization in 6×SSC at about 45° C., followed by one or more washes in 0.2×SSC, 0.1% (w/v) SDS at 60° C. Preferably, stringent hybridization conditions are hybridization in 6×SSC at about 45° C., followed by one or more washes in 0:2×SSC, 0.1% (w/v) SDS at 65° C. Particularly preferred stringency conditions (and the conditions that should be used if the practitioner is uncertain about what conditions should be applied to determine if a molecule is within a hybridization limitation of the invention) are 0.5 molar sodium phosphate, 7% (w/v) SDS at 65° C., followed by one or more washes at 0.2×SSC, 1% (w/v) SDS at 65° C. Preferably, an isolated nucleic acid molecule of the invention that hybridizes under stringent conditions to the sequence of SEQ ID NO:1, 2, 4, 6, 7, 9, 11, or 12, corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding protein, and can further include non-coding regulatory sequences and introns.

A “non-essential” amino acid residue is a residue that can be altered from the wild-type sequence of (e.g., the sequence of SEQ ID NO:3, 5, 8, 10 or 13) without abolishing or, more preferably, without substantially altering a biological activity, whereas an “essential” amino acid residue results in such a change. For example, amino acid residues that are conserved among the polypeptides of the present invention, e.g., those present in the conserved potassium channel domain are predicted to be particularly non-amenable to alteration, except that amino acid residues in transmembrane domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains 35 (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in protein is preferably replaced with another amino acid residue from the same side chain family. Alternatively, in another embodiment, mutations can be introduced randomly along all or part of coding sequences, such as by saturation mutagenesis, and the resultant mutants can be screened for biological activity to identify mutants that retain activity. Following mutagenesis of SEQ ID NO:1, 2, 4, 6, 7, 9, 11, or 12, the encoded proteins can be expressed recombinantly and the activity of the protein can be determined.

As used herein, a “biologically active portion” of protein includes fragment of protein that participate in an interaction between molecules and non-molecules. Biologically active portions of protein include peptides comprising amino acid sequences sufficiently homologous to or derived from the amino acid sequences of the protein, e.g., the amino acid sequences shown in SEQ ID NO: 3, 5, 8, 10 and 13, which include fewer amino acids than the full length protein, and exhibit at least one activity of protein. Typically, biologically active portions comprise a domain or motif with at least one activity of the protein, e.g., the ability to modulate membrane excitability, intracellular ion concentration, membrane polarization, and action potential.

A biologically active portion of protein can be a polypeptide that is, for example, 50,100, 150, 200, 250, 300, 350, 400, 450, 500 or more amino acids in length of SEQ ID NO: 3, 5, 8, 10 or 13. Biologically active portions of protein can be used as targets for developing agents that modulate-mediated activities, e.g., biological activities described herein.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nim.nih.gov>.

Polypeptides expressed by the vector of the present invention can have amino acid sequences sufficiently or substantially identical to the amino acid sequences of SEQ ID NO:3, 5, 8, 10, or 13. The terms “sufficiently identical” or “substantially identical” are used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%. 91%, 92%, 93%, 94%, 95%, 96%, 97%, 98% or 99% identity are defined herein as sufficiently or substantially identical.

The expression vector of the present application comprises a nucleic acid sequence encoding a bidirectional hydrogenase enzyme protein complex.

The nucleic acid sequence preferably encodes the bidirectional hydrogenase enzyme protein complex of Synechocystis sp. PCC 6803, which is encoded by the hox operon illustrated generally in FIG. 2.

The nucleic acid sequence of the hox operon of the present application is shown in SEQ ID NO: 1. The sequence is approximately 6532 nucleotides in length. The operon contains eight coding sequences: SEQ ID NO's: 1, 2, 4, 6, 7, 9, 11 and 12.

SEQ ID NO:2 (nucleotides 31 to 429 of SEQ ID NO: 1) is approximately 399 nucleotides in length and encodes a 133 amino acid, of the 522 nucleotide (174 amino acid) diaphorase, NADH dehydrogenase I, chain E (SEQ ID NO: 3) designated hoxE.

SEQ ID NO:4 (nucleotides 627 to 2228 of SEQ ID NO: 1) is approximately 1602 nucleotides in length and encodes a 533 amino acid NADH dehydrogenase I, chain F (SEQ ID NO: 5) designated hoxF.

SEQ ID NO:6 (nucleotides 2269 to 2907 of SEQ ID NO: 1) is approximately 639 nucleotides in length and encodes an unknown protein that shares 28.1% identity to viral regulatory protein E2, involved in transcriptional regulation and DNA replication.

SEQ ID NO:7 (nucleotides 2934 to 3650 of SEQ ID NO:1) is approximately 717 nucleotides in length and encodes a 238 amino acid diaphorase, NAD-reducing hydrogenase gamma sub unit (SEQ ID NO:8) designated hoxU.

SEQ ID NO:9 (nucleotides 3696 to 4244 of SEQ ID NO:1) is approximately 549 nucleotides in length and encodes a 182 amino acid NAD-reducing hydrogenase delta sub unit (SEQ ID NO: 10) designated hoxY.

SEQ ID NO:11 (nucleotides 4560 to 5009 of SEQ ID NO:1) is approximately 450 nucleotides in length and encodes an unknown protein that shares 32.8% identity to a Thermus theromophilus HB27 protein, also of unknown function.

SEQ ID NO:12 (nucleotides 5099 to 6523 of SEQ ID NO:1) is approximately 1425 nucleotides in length and encodes a 474 amino acid NAD-reducing hydrogenase beta sub unit (SEQ ID NO: 13) designated hoxH.

Further nucleic acid molecules incorporated into the expression vector of the present invention are described below.

In one embodiment, the expression vector of the invention comprises nucleic acid molecule comprising the nucleotide sequence of SEQ ID NO:1, or a portions or fragment thereof. In one embodiment, the expression vector comprises a nucleic acid molecule comprising a nucleotide sequence encoding the polypeptides of SEQ ID NO's: 3, 5, 8, and 13 (the Synochocystis sp. PCC6803 pentameric hydrogenase protein complex sub units). In a preferred embodiment the expression vector comprises a nucleic acid molecule comprising a nucleotide sequence of SEQ ID NO:'s 2, 4, 7, 9 and 12 (the HoxEFUYH coding regions). In an alternative embodiment the expression vector comprises a nucleic acid molecule comprising a nucleotide sequence of SEQ ID NO:'s 2, 4, 6, 7, 9, 11 and 12. In yet another embodiment, the expression vector comprises a nucleotide sequence comprising fragments of SEQ ID NO:1, preferably the fragments are biologically active fragments, i.e. having hydrogenase activity.

In another embodiment, the expression vector comprises a nucleic acid sequence that is the complement of the nucleotide sequences shown in any of SEQ ID NO's:1, 2, 4, 6, 7, 9, 11 and 12, or portions or fragments thereof. In other embodiments, expression vector comprises a nucleic acid sequence that is sufficiently complementary to the nucleotide sequence shown in any of SEQ ID NO's:1, 2, 4, 6, 7, 9, 11 and 12 such that it can hybridize to the nucleotide sequences shown in any of SEQ ID NO's:1, 2, 4, 6, 7, 9, 11 and 12 respectively, thereby forming stable duplexes.

In one embodiment, the expression vector comprises a nucleic acid sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence shown in SEQ ID NO:1, or portions or fragments thereof.

In one embodiment the expression vector comprises a nucleic acid sequence which encodes a naturally occurring allelic variant of a polypeptide comprising the amino acid sequence shown in SEQ ID NO: 3, 5, 8, 10 and 13. Allelic variants of the hydrogenase sub units shown in SEQ ID NO: 3, 5, 8, 10 or 13 include both functional and hydrogenase sub units of hoxE, hoxF, hoxU, hoxY or hoxH. Functional allelic variants are naturally occurring amino acid sequence variants of the hydrogenase sub. units of hoxE, hoxF, hoxU, hoxY or hoxH shown in SEQ ID NO: 3, 5, 8, 10 and 13 that maintain hydrogenase activity. Functional allelic variants will typically contain only conservative substitution of one or more amino acids of SEQ ID NO: 3, 5, 8, 10 or 13, or substitution, deletion or insertion of non-critical residues in non-critical regions of the protein. Non-functional allelic variants are naturally occurring amino acid sequence variants of SEQ ID NO: 3, 5, 8, 10 or 13 that do not have hydrogenase activity. Non-functional allelic variants will typically contain a non-conservative substitution, a deletion, or insertion or premature truncation of the amino acid sequence of SEQ ID NO: 3, 5, 8, 10 or 13, or a substitution, insertion or deletion in critical residues or critical regions. Nucleic acid molecules corresponding to natural allelic variants and homologues of the hydrogenase nucleic acid molecules of the invention can be isolated based on their homology to the nucleic acid molecules of the invention using the nucleotide sequences described in SEQ ID NO:1, 2, 4, 6, 7, 9, 11 or 12, or a portion thereof, as a hybridization probe under stringent hybridization conditions.

In a further embodiment the expression vector comprises a nucleic acid molecule a represented by the nucleic acid sequence in SEQ ID NO:2, or a variant nucleic acid molecule that hybridises to SEQ ID NO: 2 and encodes a polypeptide that has diaphorase activity.

In a further embodiment the expression vector comprises a nucleic acid molecule a represented by the nucleic acid sequence in SEQ ID NO:4, or a variant nucleic acid molecule that hybridises to SEQ ID NO: 4 and encodes a polypeptide that has NADH dehydrohgenase I activity.

In a further embodiment the expression vector comprises a nucleic acid molecule a represented by the nucleic acid sequence in SEQ ID NO:7, or a variant nucleic acid molecule that hybridises to SEQ ID NO: 7 and encodes a polypeptide that has NAD reducing hydrogenase gamma activity.

In a further embodiment the expression vector comprises a nucleic acid molecule a represented by the nucleic acid sequence in SEQ ID NO:9, or a variant nucleic acid molecule that hybridises to SEQ ID NO: 9 and encodes a polypeptide that has NAD reducing hydrogenase delta activity.

In a further embodiment the expression vector comprises a nucleic acid molecule a represented by the nucleic acid sequence in SEQ ID NO:12, or a variant nucleic acid molecule that hybridises to SEQ ID NO: 12 and encodes a polypeptide that has NAD reducing hydrogenase beta activity.

In a further embodiment the expression vector comprises a nucleic acid molecule comprising the nucleotide sequence of SEQ ID NO:2, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:2, or portions or fragments thereof. In another embodiment, the expression vector comprises a nucleic acid molecule comprising the nucleotide sequence of SEQ ID NO:2 or portions or fragments thereof, and at least one nucleotide sequence of SEQ ID NO: 4, 6, 7, 9, 11 or 12, or portions or fragments thereof. In another embodiment, the expression vector comprises a nucleic acid molecule comprising the nucleotide sequence of SEQ ID NO:2, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100% homologous to the entire length of the nucleotide sequence of SEQ ID NO: 4, 6, 7, 9, 11 or 12, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100% homologous to the entire length of the nucleotide sequence of SEQ ID NO:2, or portions or fragments thereof, and at least one nucleotide sequence of SEQ ID NO: 4, 6, 7, 9, 11 or 12, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:2, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO: 4, 6, 7, 9, 11 or 12, or portions or fragments thereof.

In another embodiment, the expression vector comprises a nucleic acid molecule comprising a nucleotide sequence that encodes the polypeptide of SEQ ID NO: 3 (the hoxE protein subunit of the Synochocystis sp. PCC6803 pentameric hydrogenase protein complex), or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 3, or portions or fragments thereof. In another embodiment, the expression vector comprises a nucleic acid molecule comprising a nucleotide sequence that encodes the polypeptide of SEQ ID NO: 3, or portions or fragments thereof, and a nucleotide sequence which encodes at least one of the polypeptides of SEQ ID NO: 5, 8, 10 or 13, or portions or fragments thereof. In another embodiment, the expression vector comprises a nucleic acid molecule comprising a nucleotide sequence that encodes the polypeptide of SEQ ID NO: 3, or portions or fragments thereof, and at least one nucleotide sequence which encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 5, 8, 10 or 13, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide SEQ ID NO: 3, or portions or fragments thereof, and a nucleotide sequence which encodes at least one of the polypeptides of SEQ ID NO: 5, 8, 10 or 13, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 3, or portions or fragments thereof, and at least one nucleotide sequence which encodes a polypeptide which is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 5, 8,10 or 13, or portions or fragments thereof.

In a further embodiment the expression vector comprises a nucleic acid molecule comprising the nucleotide sequence of SEQ ID NO:4, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:4, or portions or fragments thereof. In another embodiment, the expression vector comprises a nucleic acid molecule comprising the nucleotide sequence of SEQ ID NO:4 or portions or fragments thereof, and at least one nucleotide sequence of SEQ ID NO: 2, 6, 7, 9, 11 or 12, or portions or fragments thereof. In another embodiment, the expression vector comprises a nucleic acid molecule comprising the nucleotide sequence of SEQ ID NO:4, or portions or fragments thereof, and at least one nucleotide sequence that is at least about: 60%, 65%, 70%,:75%, 80%, 85%, 90%, 91%; 92%, 93%, 94%, 95%, 96%, 97%, 98%, 99% or. 100% homologous to the entire length of the nucleotide sequence of SEQ ID NO: 2, 6, 7, 9, 11 or 12, or portions or fragments thereof. In another embodiment, the expression vector comprises a nucleic acid molecule comprising a nucleotide sequence that is at least about: 60%, 65%, 70%, 75%, 80%, 85%, 95%, 91%, 92%, 93%, 94%, 95%, 96%, 97%, 98%, 990% or 100% homologous to the entire length of the nucleotide sequence of SEQ ID NO:4, or portions or fragments thereof, and at least one nucleotide sequence of SEQ ID NO: 2, 6, 7, 9, 11 or 12, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:4, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO: 2, 6, 7, 9, 11 or 12, or portions or fragments thereof.

In another embodiment, the expression vector comprises a nucleic acid molecule comprising a nucleotide sequence that encodes the polypeptide of SEQ ID NO: 5 (the hoxF protein subunit of the Synochocystis sp. PCC6803 pentameric hydrogenase protein complex), or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 5, or portions or fragments thereof. In another embodiment, the expression vector comprises a nucleic acid molecule comprising a nucleotide sequence that encodes the polypeptide of SEQ ID NO: 5, or portions or fragments thereof, and a nucleotide sequence which encodes at least one of the polypeptides of SEQ ID NO: 3, 8, 10 or 13, or portions or fragments thereof. In another embodiment, the expression vector comprises a nucleic acid molecule comprising a nucleotide sequence that encodes the polypeptide of SEQ ID NO: 5, or portions or fragments thereof, and at least one nucleotide sequence which encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 3, 8, 10 or 13, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide SEQ ID NO: 5, or portions or fragments thereof, and a nucleotide sequence which encodes at least one of the polypeptides of SEQ ID NO: 3, 8, 10 or 13, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 5, or portions or fragments thereof, and at least one nucleotide sequence which encodes a polypeptide which is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 3, 8, 10 or 13, or portions or fragments thereof.

In a further embodiment the expression vector comprises a nucleic acid molecule comprising the nucleotide sequence of SEQ ID NO:7, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:7, or portions or fragments thereof. In another embodiment, the expression vector comprises a nucleic acid molecule comprising the nucleotide sequence of SEQ ID NO:7 or portions or fragments thereof, and at least one nucleotide sequence of SEQ ID NO: 2, 4, 6, 9, 11 or 12, or portions or fragments thereof. In another embodiment, the expression vector comprises a nucleic acid molecule comprising the nucleotide sequence of SEQ ID NO:7, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100% homologous to the entire length of the nucleotide sequence of SEQ ID NO: 2, 4, 6, 9, 11 or 12, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100% homologous to the entire length of the nucleotide sequence of SEQ ID NO:7, or portions or fragments thereof, and at least one nucleotide sequence of SEQ ID NO: 2, 4, 6, 9, 11 or 12, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:7, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO: 2, 4, 6, 9, 11 or 12, or portions or fragments thereof.

In another embodiment, the expression vector comprises a nucleic acid molecule comprising a nucleotide sequence that encodes the polypeptide of SEQ ID NO: 8 (the hoxU protein subunit of the Synochocystis sp. PCC6803 pentameric hydrogenase protein complex), or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 8, or portions or fragments thereof. In another embodiment, the expression vector comprises a nucleic acid molecule comprising a nucleotide sequence that encodes the polypeptide of SEQ ID NO: 8, or portions or fragments thereof, and a nucleotide sequence which encodes at least one of the polypeptides of SEQ ID NO: 3, 5, 10 or 13, or portions or fragments thereof. In another embodiment, the expression vector comprises a nucleic acid molecule comprising a nucleotide sequence that encodes the polypeptide of SEQ ID NO: 8, or portions or fragments thereof, and at least one nucleotide sequence which encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 3, 5, 10 or 13, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide SEQ ID NO: 8, or portions or fragments thereof, and a nucleotide sequence which encodes at least one of the polypeptides of SEQ ID NO: 3, 5, 10 or 13, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 8, or portions or fragments thereof, and at least one nucleotide sequence which encodes a polypeptide which is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 3, 5, 10 or 13, or portions or fragments thereof.

In a further embodiment the expression vector comprises a nucleic acid molecule comprising the nucleotide sequence of SEQ ID NO:9, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:9, or portions or fragments thereof. In another embodiment, the expression vector comprises a nucleic acid molecule comprising the nucleotide sequence of SEQ ID NO:9 or portions or fragments thereof, and at least one nucleotide sequence of SEQ ID NO: 2, 4, 6, 7, 11 or 12, or portions or fragments thereof. In another embodiment, the expression vector comprises a nucleic acid molecule comprising the nucleotide sequence of SEQ ID NO:9, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100% homologous to the entire length of the nucleotide sequence of SEQ ID NO: 2, 4, 6, 7, 11 or 12, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100% homologous to the entire length of the nucleotide sequence of SEQ ID NO:9, or portions or fragments thereof, and at least one nucleotide sequence of SEQ ID NO: 2, 4, 6, 7, 11 or 12, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:9, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO: 2, 4, 6, 7, 11 or 12, or portions or fragments thereof.

In another embodiment, the expression vector comprises a nucleic acid molecule comprising a nucleotide sequence that encodes the polypeptide of SEQ ID NO: 10 (the hoxY protein subunit of the Synochocystis sp. PCC6803 pentameric hydrogenase protein complex), or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 10, or portions or fragments thereof. In another embodiment, the expression vector comprises a nucleic acid molecule comprising a nucleotide sequence that encodes the polypeptide of SEQ ID NO: 10, or portions or fragments thereof, and a nucleotide sequence which encodes at least one of the polypeptides of SEQ ID NO: 3, 5, 8 or 13, or portions or fragments thereof. In another embodiment, the expression vector comprises a nucleic acid molecule comprising a nucleotide sequence that encodes the polypeptide of SEQ ID NO: 10, or portions or fragments thereof, and at least one nucleotide sequence which encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 3, 5, 8 or 13, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide SEQ ID NO: 10, or portions or fragments thereof, and a nucleotide sequence which encodes at least one of the polypeptides of SEQ ID NO: 3, 5, 8 or 13, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 10, or portions or fragments thereof, and at least one nucleotide sequence which encodes a polypeptide which is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 3, 5, 8 or 13, or portions or fragments thereof.

In a further embodiment the expression vector comprises a nucleic acid molecule comprising the nucleotide sequence of SEQ ID NO:12, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:12, or portions or fragments thereof. In another embodiment, the expression vector comprises a nucleic acid molecule. comprising the nucleotide sequence of SEQ ID NO:12 or portions or fragments thereof, and at least one nucleotide sequence of SEQ ID NO: 2, 4, 6, 7, 9 or 11, or portions or fragments thereof. In another embodiment, the expression vector comprises a nucleic acid molecule comprising the nucleotide sequence of SEQ ID NO:12, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100% homologous to the entire length of the nucleotide sequence of SEQ ID NO: 2, 4, 6, 7, 9 or 11, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100% homologous to the entire length of the nucleotide sequence of SEQ ID NO:12, or portions or fragments thereof, and at least one nucleotide sequence of SEQ ID NO: 2, 4, 6, 7, 9 or 11, or portions or fragments thereof. In another embodiment, the expression vector comprises a nucleic acid molecule comprising a nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO:12, or portions or fragments thereof, and at least one nucleotide sequence that is at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence of SEQ ID NO: 2, 4, 6, 7, 9 or 11, or portions or fragments thereof.

In another embodiment, the expression vector comprises a nucleic acid molecule comprising a nucleotide sequence that encodes the polypeptide of SEQ ID NO: 13 (the hoxH protein subunit of the Synochocystis sp. PCC6803 pentameric hydrogenase protein complex), or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 13, or portions or fragments thereof. In another embodiment, the expression vector comprises a nucleic acid molecule comprising a nucleotide sequence that encodes the polypeptide of SEQ ID NO: 13, or portions or fragments thereof, and a nucleotide sequence which encodes at least one of the polypeptides of SEQ ID NO: 3, 5, 8 or 10, or portions or fragments thereof. In another embodiment, the expression vector comprises a nucleic acid molecule comprising a nucleotide sequence that encodes the polypeptide of SEQ ID NO: 13, or portions or fragments thereof, and at least one nucleotide sequence which encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 3, 5, 8 or 10, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length of the polypeptide SEQ ID NO: 13, or portions or fragments thereof, and a nucleotide sequence which encodes at least one of the polypeptides of SEQ ID NO: 3, 5, 8 or 10, or portions or fragments thereof. In another embodiment, the expression vector comprises a nucleic acid molecule comprising a nucleotide sequence that encodes a polypeptide that is at least about: 60%, 65%, 70%, 75%, 80%, 85%, 90%, 91%, 92%, 93%, 94%, 95%, 96%, 97%, 98%, 99% or 100%, homologous to the entire length the polypeptide of SEQ ID NO: 13, or portions or fragments thereof, and at least one nucleotide sequence which encodes a polypeptide which is at least about: 60%, 65%, 70%, 75%, 80%, 85%, 90%, 91%, 92%, 93%, 94%, 95%, 96%, 97%, 98%, 99% or 100%, homologous to the entire length of the polypeptide of SEQ ID NO: 3, 5, 8 or 10, or portions or fragments thereof.

In another embodiment the expression vector comprises a nucleic acid molecule as described previously, comprising specific changes in the nucleotide sequence so as to optimize codons and mRNA secondary structure for translation in the host cell. Preferably, the codon usage of the nucleic acid is adapted for expression in the host cell, for example codon optimisation can be achieved using Calcgene, Hale, R S and Thomas G. Protein Exper. Purif. 12, 185-188 (1998), UpGene, Gao, W et al. Biotechnol. Prog. 20, 443-448 (2004), or Codon Optimizer, Fuglsang, A. Protein Exper. Purif. 31, 247-249 (2003). Amending the nucleic acid according to the preferred codon optimization can be achieved by a number of different experimental protocols, including, modification of a small number of codons, Vervoort et al. Nucleic Acids Res. 25: 2069-2074 (2000), or rewriting a large section of the nucleic acid sequence, for example, up to 1000 bp of DNA, Hale, R S and Thomas G. Protein Exper. Purif. 12, 185-188 (1998). Rewriting of the nucleic acid sequence can be achieved by recursive PCR, where the desired sequence is produced by the extension of overlapping oligonucleotide primers, Prodromou and Pearl, Protein Eng. 5: 827-829 (1992). Rewriting of larger stretches of DNA may require up to three consecutive rounds of recursive PCR, Hale, R S and Thomas G. Protein Exper. Purif. 12, 185-188 (1998), Te'o et al, FEMS Microbiol. Lett. 190: 13-19, (2000).

Alternatively, the level of cognent tRNA can be elevated in the host cell. This elevation can be achieved by increasing the copy number of the respective tRNA gene, for example by inserting into the host cell the relevant tRNA gene on a compatible multiple copy plasmid, or alternatively inserting the tRNA gene into the expression vector itself. When using an E. coli expression system, E. coli host cells having enhanced expression of argU expression (for recognition of AGG/AGA) may be employed. In addition, host cells comprising tRNA genes for ilex (for recognition of AUA), leuW (for recognition of CUA), proL (for recognition of CCC) or glyT (for recognition of GGA) may also be employed, Brinkmann et al. Genes, 85, 109-114, (1989), Kane F J. Curr. Opin. Biotechnol. 6:494-500 (1995), Rosenburg et al, J. Bacteriol. 175, 716-722, (1993), Siedel et al, Biochemistry, 31, 2598-2608, (1992).

In another embodiment the expression vector comprises a nucleic acid molecule as described previously, comprising specific changes in the nucleotide sequence so as to optimize expression, activity or functional life of the bidirectional hydrogenase. Preferably, the bidirectional hydrogenase nucleic acids described previously are subjected to genetic manipulation and disruption techniques. Various genetic manipulation and disruption techniques are known in the art including, but not limited to, DNA Shuffling (U.S. Pat. No. 6,132,970, Punnonen J et al, Science & Medicine, 7(2): 38-47, (2000), U.S. Pat. No. 6,132,970), serial mutagenesis and screening. One example of mutagenesis is error-prone PCR, whereby mutations are deliberately introduced during PCR through the use of error-prone DNA polymerases and reaction conditions as described in US 2003152944, using for example commercially available kits such as The GeneMorph® II kit (Stratagene®, US). Randomized DNA sequences are cloned into expression vectors and the resulting mutant libraries screened for altered or improved protein activity.

### Preparation of Hox Expression Vectors

A man of skill in the art will be aware of the molecular techniques available for the preparation of expression vectors.

The nucleic acid molecule for incorporation into the expression vector of the invention, as described above, can be prepared by synthesizing nucleic acid molecules using mutually priming oligonucleotides and the nucleic acid sequences described herein.

A number of molecular techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the nucleic acid molecule to the expression vector. In one embodiment, the nucleic acid molecule is generated by restriction endonuclease digestion as described earlier. Preferably, the nucleic acid molecule is treated with bacteriophage T4 DNA polymerase or E. coli DNA polymerase 1, enzymes that remove protruding, 3′-single-stranded termini with their 3′-5′-exonucleolytic activities, and fill in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a nucleic acid molecule carrying polymeric linker sequences at its ends. These nucleic acid molecules are then cleaved with the appropriate restriction enzyme and ligated to an expression vector that has been cleaved with an enzyme that produces termini compatible with those of the nucleic acid molecule.

Alternatively, a vector comprising ligation-independent cloning (LIC) sites can be employed. The required PCR amplified nucleic acid molecule can then be cloned into the LIC vector without restriction digest or ligation (Aslanidis and de Jong, Nucl. Acid. Res. 18, 6069-6074, (1990), Haun, et al, Biotechniques 13, 515-518 (1992).

In order to isolate and/or modify the nucleic acid molecule of interest for insertion into the chosen plasmid, it is preferable to use PCR. Appropriate primers for use in PCR preparation of the sequence can be designed to isolate the required coding region of the nucleic acid molecule, add restriction endonuclease or LIC sites, place the coding region in the desired reading frame.

In a preferred embodiment a nucleic acid molecule for incorporation into an expression vector of the invention, is prepared by the use of the polymerase chain reaction as disclosed by Saiki et al (1988) Science 239, 487-491, using appropriate oligonucleotide primers. The coding region is amplified, whilst the primers themselves become incorporated into the amplified sequence product. In a preferred embodiment the amplification primers contain restriction endonuclease recognition sites which allow the amplified sequence product to be. cloned into an appropriate vector.

Preferably, the nucleic acid molecule of SEQ ID NO:1 is obtained by PCR and introduced into an expression vector using restriction endonuclease digestion and ligation, a technique which is well known in the art. More preferably the nucleic acid molecule of SEQ ID NO:1 is introduced to pET-17b expression vector and is operatively linked to a T7 promoter.

Alternatively, the nucleic acid molecule of SEQ ID NO:1 is introduced into an expression vector by yeast homologous recombination (Raymon et al., Biotechniques. 26(1): 134-8, 140-1, 1999).

The expression vectors of the invention can contain a single copy of the nucleic acid molecule described previously, or multiple copies of the nucleic acid molecule described previously.

Preferably, the expression vector of the present invention is a pET-17b expression vector (3306 bp) comprising the bidirectional hydrogenase of SEQ ID NO:1 (6532 bp) as illustrated in FIG. 4.

### Host Cells

“Purified preparation of cells,” as used herein, refers to, in the case of cultured cells or microbial cells, a preparation of at least 10%, and more preferably, 50% of the subject cells.

“Host cell” and “recombinant host cell”, as used herein, are used interchangeably. The terms refer to the particular subject cell and also to the progeny or potential progeny of such a cell. Because certain modifications may occur in succeeding generations due to either mutation or environmental influences, such progeny may not, in fact, be identical to the parent cell, but are still included within the scope of the term as used herein.

Another aspect the invention provides a host cell for use in the expression system of the present invention which comprises an expression vector, comprising a nucleic acid molecule described herein, e.g., the Hox operon of SEQ ID NO:1, or portions or fragments thereof. In an alternative embodiment the host cell comprises an expression vector of the present invention, comprising a nucleic acid molecule described herein, e.g., the Hox operon of SEQ ID NO:1, or portions or fragments thereof, the vector further comprising sequences which allow it to homologously recombine into a specific site of the host cell's genome.

The host cell for use in the expression system of the present invention may be an aerobic cell or alternatively a facultative anaerobic cell. Preferably, the cell is a bacterial cell. Alternatively, the cell may be a yeast cell (e.g. Saccharomyces, Pichia), an algae cell, an insect cell, or a plant cell.

Bacterial host cells include Gram-positive and Gram-negative bacteria. Suitable bacterial host cells include, but are not limited to the Gram-negative bacteria, for example a bacterium of the family Enterobacteria, most preferably Escherichia coli. E. coli is the most preferred bacterial host cells for the present invention. Expression in E. coli offers numerous advantages over other expression systems, particularly low development costs and high production yields. Cells suitable for high protein expression include, for example, E. coli W3110, the B strains of E. coli. E. coli BL21, BL21 (DE3), and BL21 (DE3) pLysS, pLysE, DH1, DH41, DH5, DH51, DH51F′, DH51MCR, DH10B, DH10B/p3, DH1 IS, C600, HB101, JM101, JM105, JM109, JM110, K38, RR1, Y1088, Y1089, CSH18, ER1451, ER1647 are particularly suitable for expression. E. coli K12 strains are also preferred as such strains are standard laboratory strains, which are non-pathogenic, and include NovaBlue, JM109 and DH5a (Novogen®), E. coli K12 RV308, E. coli K12 C600, E. coli HB101, see, for example, Brown, Molecular Biology Labfax (Academic Press (1991)).

Alternatively, Enterobacteria from the genus Salmonella, Shigella, Enterobacter, Serratia, Proteus and Erwinia. Other prokaryotic host cells include Serratia, Pseudomonas, Caulobacter, or Cyanobacteria, for example bacteria from the genus Synechocystis or Synechococcus, more particularly Synechocystis sp. PCC 6803 or Synechococcus sp PCC 6301. Alternatively, the host cell may be of the genus Bacillus, for example Bacillus brevis or Bacillus subtilis, Bacillus thuringienesis. Alternatively, the host cell may be of the genus Lactococcus, for example Lactococcus lactis. Alternatively, the bacterial cell is of the actinomycetes family, more particularly from the genus Streptomyces, Rhodococcus, Corynebacterium, Mycobacterium. More particularly, Streptomyces lividans, Streptomyces ambofaciens, Streptomyces fradiae, Streptomyces griseofuscus, Rhodococcus erythropolis, Corynebacterium gluamicum, Mycobacterium smegmatis.

Standard techniques for propagating vectors in prokaryotic hosts are well-known to those of skill in the art (see, for example, Ausubel et al. Short Protocols in Molecular Biology 3rd Edition (John Wiley & Sons 1995)).

To maximize recombinant protein expression in E. coli, the expression vectors of the invention may express the nucleic acid molecule incorporated therein in a host bacteria with an impaired capacity to proteolytically cleave the recombinant protein (Gottesman, S., (1990) Gene Expression Technology: Methods in Enzymology 185, Academic Press, San Diego, Calif., 119-128). Alternatively, the nucleic acid molecule incorporated into an expression vector of the invention, can be attenuated so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

### Host Cell Transformation

The expression vector of the present invention can be introduced into host cells by conventional transformation or transfection techniques.

“Transformation” and “transfection”, as used herein, refer to a variety of techniques known in the art for introducing foreign nucleic acids into a host cell. Transformation of appropriate host cells with an expression vector of the present invention is accomplished by methods known in the art and typically depends on both the type of vector and host cell. Said techniques include, but are not limited to calcium phosphate or calcium chloride co-precipitation, DEAE-dextran-mediated transfection, lipofection, chemoporation or electroporation.

Techniques known in the art for the transformation of bacterial host cells are disclosed in for example, Sambrook et al (1989) Molecular Cloning, A Laboratory Manual, Cold Spring Harbor Laboratory, Cold Spring Harbor, N.Y; Ausubel et al (1987) Current Protocols in Molecular Biology, John Wiley and Sons, Inc., NY; Cohen et al (1972) Proc. Natl. Acad. Sci. USA 69, 2110; Luchansky et al (1988) Mol. Microbiol. 2, 637-646. All such methods are incorporated herein by reference.

Successfully transformed cells, that is, those cells containing the expression vector of the present invention, can be identified by techniques well known in the art. For example, cells transfected with the expression vector of the present invention can be cultured to produce the bidirectional hydrogenase protein complex. Cells can be examined for the presence of the expression vector DNA by techniques well known in the art. Alternatively, the presence of the bidirectional hydrogenase protein complex, or portion and fragments thereof can be detected using antibodies which hybridize thereto.

In a preferred embodiment the invention comprises a culture of transformed host cells. Preferably the culture is clonally homogeneous.

The host cell can contain a single copy of the expression vector described previously, or alternatively, multiple copies of the expression vector.

### Hydrogen Production

A host cell transformed with an expression vector of the invention, comprising a nucleic acid molecule as described previously, can be used to produce (i.e., express) a polypeptide having hydrogenase activity.

Preferably, the present invention comprise an expression system for the large scale production of hydrogen, utilizing a nucleic acid coding sequence of the present invention, encoding a bidirectional hydrogenase protein. Preferably the expression system is an E. coli expression system.

Transformed host cells of the invention are grown or cultured in the manner with which the skilled worker is familiar, depending on the host organism. As a rule, host cells are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen. The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the organisms as described above by processes known to the skilled worker, for example by extraction, distillation, crystallization, if appropriate precipitation with salt, and/or chromatography. To this end, the host cells can advantageously be disrupted beforehand. In this process, the pH value is advantageously kept between pH 4 and 12, preferably between pH 6 and 9, especially preferably between pH 7 and 8.

An overview of known cultivation methods can be found in the textbook by Chmiel (Bioprozeβtechnik 1. Einführung in die Bioverfahrenstechnik [Bioprocess technology 1. Introduction to Bioprocess technology] (Gustav Fischer Verlag, Stuttgart, 1991)) or in the textbook by Storhas (Bioreaktoren und periphere Einrichtungen [Bioreactors and peripheral equipment] (Vieweg Verlag, Brunswick/Wiesbaden, 1994)).

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing host cells usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., more preferably at from 25 to 37° C., more preferably from 35 to 37° C., more preferably at 37° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of vector it is possible to add to the medium suitable substances having a selective effect, for example antibiotics. Aerobic conditions are maintained by introducing oxygen or oxygen-containing gas mixtures such as, for example, ambient air into the culture. The temperature of the culture is normally 20° C. to 45° C. and preferably 25° C. to 40° C. The culture is continued until formation of the desired product is at a maximum. This aim is normally achieved within 10 to 160 hours.

The fermentation broths obtained in this way, in particular those comprising polyunsaturated fatty acids, usually contain a dry mass of from 7.5 to 25% by weight.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

However, the fermentation broth can also be thickened or concentrated without separating the cells, using known methods such as, for example, with the aid of a rotary evaporator, thin-film evaporator, falling-film evaporator, by reverse osmosis or by nanofiltration. Finally, this concentrated fermentation broth can be processed to obtain the fatty acids present therein.

Preferably, transformed host cells are cultured so that a bidirectional hydrogenase protein complex is produced. Preferably, cells are cultured in conditions capable of inducing hydrogen production by the host cell.

Transformed host cells can be cultured using a batch fermentation, particularly when large scale hydrogen production of hydrogen using the bidirectional hydrogenase expression system of the present invention is required. Alternatively, a fed batch and/or continuous culture can be used to generate a yield of hydrogen from host cells transformed with the bidirectional hydrogenase expression system of the present invention.

Transformed host cells can be cultured in aerobic or anaerobic conditions. In aerobic conditions, preferably, oxygen is continuously removed from the culture medium, by for example, the addition of reductants or oxygen scavengers, or, by purging the reaction medium with neutral gases.

Techniques known in the art for the large scale culture of host cells are disclosed in for example, Bailey and Ollis (1986) Biochemical Engineering Fundamentals, McGraw-Hill, Singapore; or Shuler (2001) Bioprocess Engineering: Basic Concepts, Prentice Hall. All such techniques are incorporated herein by reference.

Preferably, transformed host cells are cultured in LB containing the appropriate selective antibiotic for the expression vector. The transformed host cells are incubated whist shaking at 37° C. until the OD₆₀₀ reaches 0.6 to 1.0. The culture is then stored at 4° C. overnight. The following morning, the cells are collected by centrifugation (30 seconds in a microcentrifuge). Collected cells are then be resuspended in fresh LB medium. Preferably the LB medium contains additional nutrient media. Preferably, the nutrient media is BG-11 or BG-110 media, Stanier R. Y. et al., (1971) Bacteriol. Rev. 35: 171-205.

Preferably, the bidirectional hydrogenase content of a culture of bacterial cells optimally expressing the bidirectional hydrogenase coding sequence of the present invention is at least 100 nmol/l culture of whole cells, preferably at least 150 nmol/l culture of whole cells more preferably almost 250 nmol/l culture of whole cells, still more preferably about 500 nmol/l culture of whole cells and most preferably about 1000 nmol/l. Typically the bidirectional hydrogenase content is around 200 nmol/l culture of whole cells.

The host cells of the invention can be cultured in a vessel, for example a bioreactor. Bioreactors, for example fermentors, are vessels that comprise cells or enzymes and typically are used for the production of molecules on an industrial scale. The molecules can be recombinant proteins (e.g. enzymes such as hydrogenases) or compounds that are produced by the cells contained in the vessel or via enzyme reactions that are completed in the reaction vessel. Typically, cell based bioreactors comprise the cells of interest and include all the nutrients and/or co-factors necessary to carry out the reactions.

### EXAMPLES

### Example 1

### Construction of Expression Vector

The bidirectional hydrogenase protein complex coding region, SEQ ID NO:1, was generated by PCR amplification using a Synechocystis sp. PCC 6803 library as a template and oligonucleotide primers SynBamFwd: ccaatcatgg atccgctgta ttgctccttt ttgagg (SEQ ID NO:14) and SynEcoRev: ggattactga attcccgtct gaatgttttt tg (SEQ ID NO:15). The resulting gene sequence encoded SEQ ID NO:1, including BamHI and EcoRI restriction sites incorporated at the 5′ and 3′ end respectively.

The resulting PCR product was cleaved by a restriction endonuclease at the incorporated restriction sites, BamHI and EcoRI, and inserted by ligation, using T4 ligase, into expression vector pET-17b (described previously) which had also been cleaved by restriction endonuclease digestion with BamHI and EcoRI, as illustrated in FIG. 4 .

### Example 2

### Construction of Expression Vector

In an alternative example the bidirectional hydrogenase protein complex coding region SEQ ID NO:1 was generated by PCR amplification using a Synechocystis sp, PCC 6803 library as a template and oligonucleotide primers SynBamFwd: ccaatcatgg atccgctgta ttgctccttt ttgagg (SEQ ID NO:14) and SynNotRev: ggattactgc ggccgcccgt ctgaatgttt tttg (SEQ ID NO:16). The resulting gene sequence encoded SEQ ID NO:1, including BamHI and NotI restriction sites incorporated at the 5′ and 3′ end respectively.

The resulting PCR product was cleaved by restriction endonuclease at the incorporated restriction sites, BamHI and NotI, and inserted by ligation, using T4 ligase, into expression vector pET-17b (described previously) which had been cleaved by restriction endonuclease digestion with BamHI and NotI.

### Example 3

### Transformation

Each of the expression vectors described in example 1 and 2 was subsequently transformed into NovoBlue® competent cells (Novagen®, USA). 1 μl of each expression vector product and 20 μl of NovaBlue® cells were incubated on ice for 5 minutes, at 42° C. for 30 seconds, and on ice for 2 minutes. 80 μl of SOC (RT) was added and reaction mixture incubated at 37° C. for 60 minutes. Reaction mixture was then plated onto LB agar, containing 50 μl carbenicillin and left at 37° C. temperature for 20 hours.

### Vector Stability

Colonies from both EcoRI expression vector transformants and NotI expression vector transformants were selected and resuspended, 100 iμl into a 10.0 ml LB broth containing 50 μg/ml carbenicillin. The reaction mixture then cultured at 37° c. for 20 hours and shaken at 250 RPM.

To confirm presence of the pET17b-hox plasmid, plasmids were extracted from cultured isolates. Extraction of NotI plasmids achieved using MoBio® 6 Minute Mini Plasmid Extraction Kit (MO BIO Laboratories, USA). Extraction of EcoRI plasmids achieved using Qiagen® Mini Plasmid Extraction Kit (Qiagen®, Inc. USA).

Extracted plasmids were subject to restriction digest, using BamHI and EcoRI, or BamHI and NotI accordingly, and digested products were subject to gel electrophoresis on 0.6% TAE Agarose gel, at 100 V for 60 minutes. Strains containing correct sized fragments, 3.3 kb pET-17b vector and 6.4 kb hox operon nucleic acid molecule insert were detected.

### Expression of Bidirectional Pentameric Hydrogenase Protein Complex

Two isolates, one NotI and one EcoRI, containing correct sized fragments, were transfected into E. coli BL21 and BL21 (DE3)pLys5 cell lines. Specifically, a 1 ng/μl dilution of isolate cells was prepared for transfection into BL21 and BL21 (DE3)pLys5 cell lines by incubating then on ice for 5 minutes, at 42° C. for 30 seconds, and on ice again for 2 minutes. 80 μl of SOC (RT) was then added and reaction mixture incubated at 37° C. for 60 minutes. 100 μl of reaction mixture was then streaked onto LB agar plates containing 50 μg/ml carbenicillin or ampicillin and then incubated overnight at 37° C.

One colony of NotI vector transfected cells was used as an innoculum, comprising transformant colonies in 1 ml LB Broth with 50 μg/ml carbenicillin, was used to inoculate a 50 ml culture in a 250 ml flask. Similarly, one colony of EcoRI vector transfected cells was used as an innoculum. Each of the flask cultures was incubated at 37° C. and shaken at 250 RPM for 4-5 hours. Cultures were then incubated with and without protein expression stimulation (induction by adding 200 μl of 100 nM IPTG (final concentration 0.4 nM)). Cultures were then further incubated at 37° C., with shaking, for three hours. Cells were then harvested by centrifugation at 5000×g at 4° C. The cell pellets were then stored dry at 70° C. for use at a later time.

Recombinant bidirectional hydrogenase protein complex accumulated as insoluble inclusion bodies and as soluble protein. Pellets were washed once with 12.5 ml TRIS-HCl pH 8.0.

Inclusion body protein was extracted using 2 ml of Bacterial Protein Extraction Reagent (B-PER in phosphate buffer; Pierce, USA) and 40 μl of 10 mg/ml lycozyme (final concentration 200 μg/ml) to further digest the cell debris and release inclusion bodies. The “inclusion body” pellet was then dissolved in 1% SDS (1 ml), via heating, vortexing and sonification.

Soluble protein was extracted using 2 ml of B-PER reagent (Pierce, USA) and mechanical homogenization via either vortexing or pipetting. This fraction was then separated using centrifugation at 27,200×g for 1 hour, resulting in greater than 90% recovery. The soluble protein fraction was concentrated using TCA precipitation, by adding 5 ml of trichloroacetic acid/acetone (5 ml of 6N TCA or 3 ml TCA, 300 μl of TBP to total volume of 30 ml using acetone), mixed well and stored at −20° C. The mixture was then centrifuged down at 4,600×g for 1 hour and then washed with equilibrium buffer (300 μl of TBP to 29,700 μl acetone). Pellets were then resuspended in 1% SDS, again aided by heating, vortexing and sonification.

Subsequently, soluble protein and inclusion bodies isolated from both the NotI and EcoRI transformed cells, were separated according to pl and visualised using SDS-polyacrylamide gel electrophoresis (SDS-PAGE). Specifically, 10 μl of each sample (soluble protein and inclusion bodies from both NotI and EcoRI cells transformed using both DE3 and pLysS being both induced and not induced) were run on 10% SDS-PAGE gels at 150V for 65 minutes. This was followed by staining for 1 hour and destaining overnight.

Taking into account the relative position for the two bidirection hydrogen sub-units (diaphorase and native) within the resultant SDS-PAGE gel, bands were excised, washed, destained, digested with trypsin and peptides extracted, prior to identification using mass spectrometry. Results of peptide fingerprinting, using QqTOF-MS-MS, showed the presence of hoxU and hoxU subunits in the induced, DE3 NotI transformed cell line. While results for the induced, EcoRI transformed cell indicated the presence of hoxH, hoxU, hoxF and hoxY, also as inclusion bodies within both DE3 and pLysS E. coli cell lines.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1-56. (canceled)

57. An expression vector for producing a hydrogenase protein or hydrogenase protein complex, comprising the operably linked elements of:
i) a transcription promoter element;
ii) a nucleic acid molecule which encodes a polypeptide having the specific enzyme activity associated with a cyanobacteria hydrogenase; and
iii) a transcriptional terminator.

58. An expression vector according to claim 57, wherein the nucleic acid molecule is selected from the group consisting of:
i) a nucleic acid molecule comprising the nucleotide sequence of SEQ ID NO: 1;
ii) a nucleic acid molecule having at least 70% identity to the nucleotide sequence of SEQ ID NO: 1 and which encodes a polypeptide that has hydrogenase activity;
iii) a nucleic acid molecule which hybridizes to the nucleic acid sequence of SEQ ID NO:1 and which encodes a polypeptide that has hydrogenase activity; or
iv) a nucleic acid molecule comprising a nucleotide sequence that is degenerate as a result of the genetic code to the sequences of i), ii) and iii) above.

59. An expression vector according to claim 58, wherein the nucleic acid molecule is at least 80%, 85%, 90% or 95% identical to the nucleotide sequence of SEQ ID NO: 1 and which encodes a polypeptide that has hydrogenase activity.

60. An expression vector according to claim 58, wherein the nucleic acid molecule consist of the nucleotide sequence of SEQ ID NO: 1.

61. An expression vector according to claim 57, wherein the nucleic acid molecule is selected from the group consisting of:
i) a nucleic acid molecule comprising the nucleotide sequence of each of SEQ ID NO: 2, 4, 7, 9 and 12;
ii) a nucleic acid molecule comprising a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11; or
iii) a nucleic acid molecule consisting of a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11.

62. An expression vector according to claim 61, wherein the nucleic acid molecule is selected from the group consisting of:
i) a nucleic acid molecule comprising a nucleotide sequence having at least 80%, 85%, 90% or 95% identity to SEQ ID NO:2, a nucleotide sequence having at least 80%, 85%, 90% or 95% identity to SEQ ID NO:4, a nucleotide sequence having at least 80%, 85%, 90% or 95% identity to SEQ ID NO:7, a nucleotide sequence having at least 80%, 85%, 90% or 95% identity to SEQ ID NO:9 and a nucleotide sequence having at least 80%, 85%, 90% or 95% identity to SEQ ID NO:1; or
ii) a nucleic acid molecule consisting of a nucleotide sequence having at least 80%, 85%, 90% or 95% identity to SEQ ID NO:2, a nucleotide sequence having at least 80%, 85%, 90% or 95% identity to SEQ ID NO:4, a nucleotide sequence having at least 80%, 85%, 90% or 95% identity to SEQ ID NO:7, a nucleotide sequence having at least 80%, 85%, 90% or 95% identity to SEQ ID NO:9 and a nucleotide sequence having at least 80%, 85%, 90% or 95% identity to SEQ ID NO:11.

63. An expression vector according to claim 61, wherein the nucleic acid molecule consists of the nucleotide sequence of each of SEQ ID NO: 2, 4, 7, 9 and 12.

64. An expression vector according to claim 57, wherein the nucleic acid molecule is selected from the group consisting of:
i) a nucleic acid molecule comprising the nucleotide sequence of at least one of SEQ ID NO: 2, 4, 7, 9 or 12; or
ii) a nucleic acid molecule comprising the nucleotide sequence of at least one of a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11.

65. An expression vector according to claim 64, wherein the nucleic acid molecule is selected from the group consisting of:
i) the nucleotide sequence of at least one of a nucleotide sequence having at least 80%, 85%, 90 or 95% identity to SEQ ID NO:2, a nucleotide sequence having at least 80%, 85%, 90 or 95% identity to SEQ ID NO:4, a nucleotide sequence having at least 80%, 85%, 90 or 95% identity to SEQ ID NO:7, a nucleotide sequence having at least 80%, 85%, 90 or 95% identity to SEQ ID NO:9 and a nucleotide sequence having at least 80%, 85%, 90 or 95% % identity to SEQ ID NO:11.

66. An expression vector according to claim 64, wherein the nucleic acid molecule is a nucleic acid molecule represented by the nucleic acid sequence in SEQ ID NO:2, or a variant nucleic acid molecule that hybridises to SEQ ID NO: 2 and encodes a polypeptide that has diaphorase activity.

67. An expression vector according to claim 64, wherein the nucleic acid molecule is a nucleic acid molecule represented by the nucleic acid sequence in SEQ ID NO:4, or a variant nucleic acid molecule that hybridises to SEQ ID NO: 4 and encodes a polypeptide that has NADH dehydrohgenase I activity.

68. An expression vector according to claim 64, wherein the nucleic acid molecule is a nucleic acid molecule represented by the nucleic acid sequence in SEQ ID NO:7, or a variant nucleic acid molecule that hybridises to SEQ ID NO: 7 and encodes a polypeptide that has NAD reducing hydrogenase gamma activity.

69. An expression vector according to claim 64, wherein the nucleic acid molecule is a nucleic acid molecule represented by the nucleic acid sequence in SEQ ID NO:9, or a variant nucleic acid molecule that hybridises to SEQ ID NO: 9 and encodes a polypeptide that has NAD reducing hydrogenase delta activity.

70. An expression vector according to claim 64, wherein the nucleic acid molecule is a nucleic acid molecule represented by the nucleic acid sequence in SEQ ID NO:12, or a variant nucleic acid molecule that hybridises to SEQ ID NO: 12 and encodes a polypeptide that has NAD reducing hydrogenase beta activity.

71. An expression vector according to claim 57, wherein the nucleic acid molecule consists of a nucleotide sequence that encodes each polypeptide of SEQ ID NO: 3, 5, 8, and 13.

72. An expression vector according to claim 57, wherein the nucleic acid molecule comprises:
i) a first nucleotide sequence that encodes a polypeptide that is at least 70% identical to SEQ ID NO:3, 5, 8, 10 or 13; and
ii) at least one further nucleotide sequence that encodes a polypeptide that is at least 70% identical to SEQ ID NO:3, 5, 8, 10 or 13.

73. An expression vector according to claim 66 wherein the variant nucleic acid molecule hybridises under stringent hybridisation conditions.

74. An expression vector according to claim 57 wherein the transcription promoter element comprises an element that confers inducible expression on said nucleic acid molecule or variant nucleic acid molecule.

75. An expression vector according to claim 57 wherein the transcription promoter element comprises an element that confers repressible expression on said nucleic acid molecule or variant nucleic acid molecule.

76. An expression vector according to claim 57 wherein the transcription promoter element confers constitutive expression on said nucleic acid molecule or variant nucleic acid molecule.

77. An expression vector according to claim 57, wherein the expression vector includes a selectable marker.

78. An expression vector according to claim 57, wherein the expression vector comprises a translational control element.

79. An expression vector according to claim 57, wherein said translational control element is a ribosomal binding sequence.

80. An expression vector according to any preceding claim, wherein said nucleic acid molecule comprises specific changes in the nucleotide sequence so as to optimize codon usage.

81. A host cell transformed with the expression vector according to claim 57.

82. A host cell according to claim 81, wherein said cell is a bacterial cell.

83. A host cell according to claim 82, wherein said bacterial cell is a gram negative bacterial cell.

84. A host cell according to claim 83, wherein said cell is of the genus Escherichia spp.

85. A host cell according to claim 84 wherein said cell is Escherichia coli.

86. A host cell according to claim 85, wherein said cell is Escherichia coli BL21 or Escherichia coli BL21 (DE3)pLys5.

87. A host cell according to claim 82, wherein said bacterial cell is a gram positive bacterial cell.

88. A host cell according to claim 81, wherein said cell comprises a vector comprising tRNA genes.

89. A host cell according to claim 88, where are said tRNA genes encode for argU, ilex, leuW, proL or glyT.

90. A method for producing hydrogen comprising:
i) incorporating a nucleic acid molecule comprising at least one cyanobacteria hydrogenase gene into an expression vector for expression in a host cell; and
ii) transfecting a host cell with the expression vector;
wherein the resulting transfected host cell produces hydrogen.

91. A method according to claim 90, wherein said at least one hydrogenase gene is a bidirectional hydrogenase gene.

92. A method according to claim 90, wherein said cyanobacteria is of the genus Synechocystis.

93. A method according to claim 92, wherein the cyanobacteria is Synechocystis sp. PCC 6803.

94. A method according to claim 90, wherein the nucleic acid molecule is selected from the group consisting of:
i) a nucleic acid molecule comprising the nucleotide sequence of SEQ ID NO: 1;
ii) a nucleic acid molecule having at least 70% identity to the nucleotide sequence of SEQ ID NO: 1;
iii) a nucleic acid molecule which hybridizes to the nucleic acid sequence of SEQ ID NO:1; or
iv) a nucleic acid molecule comprising a nucleotide sequence that is degenerate as a result of the genetic code to the sequences of i), ii) and iii) above.

95. A method according to claim 94, wherein the nucleic acid molecule consist of the nucleotide sequence of SEQ ID NO: 1.

96. A method according to claim 90, wherein the nucleic acid molecule is selected from the group consisting of:
i) a nucleic acid molecule comprising the nucleotide sequence of each of SEQ ID NO: 2, 4, 7, 9 and 12;
ii) a nucleic acid molecule comprising a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11; or
iii) a nucleic acid molecule consisting of a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11.

97. A method according to claim 96, wherein the nucleic acid molecule consists of the nucleotide sequence of each of SEQ ID NO: 2, 4, 7, 9 and 12.

98. A method according to claim 90, wherein the nucleic acid molecule is selected from the group consisting of:
i) a nucleic acid molecule comprising the nucleotide sequence of at least one of SEQ ID NO: 2, 4, 7, 9 or 12; or
ii) a nucleic acid molecule comprising the nucleotide sequence of at least one of a nucleotide sequence having at least 70% identity to SEQ ID NO:2, a nucleotide sequence having at least 70% identity to SEQ ID NO:4, a nucleotide sequence having at least 70% identity to SEQ ID NO:7, a nucleotide sequence having at least 70% identity to SEQ ID NO:9 and a nucleotide sequence having at least 70% identity to SEQ ID NO:11.

99. A method according to claim 98, wherein the nucleic acid molecule is a nucleic acid molecule represented by the nucleic acid sequence in SEQ ID NO:2, or a variant nucleic acid molecule that hybridises to SEQ ID NO: 2 and encodes a polypeptide that has diaphorase activity.

100. A method according to claim 98, wherein the nucleic acid molecule is a nucleic acid molecule represented by the nucleic acid sequence in SEQ ID NO:4, or a variant nucleic acid molecule that hybridises to SEQ ID NO: 4 and encodes a polypeptide that has NADH dehydrohgenase I activity.

101. A method according to claim 98, wherein the nucleic acid molecule is a nucleic acid molecule represented by the nucleic acid sequence in SEQ ID NO:7, or a variant nucleic acid molecule that hybridises to SEQ ID NO: 7 and encodes a polypeptide that has NAD reducing hydrogenase gamma activity.

102. A method according to claim 98, wherein the nucleic acid molecule is a nucleic acid molecule represented by the nucleic acid sequence in SEQ ID NO:9, or a variant nucleic acid molecule that hybridises to SEQ ID NO: 9 and encodes a polypeptide that has NAD reducing hydrogenase delta activity.

103. A method according to claim 98, wherein the nucleic acid molecule is a nucleic acid molecule represented by the nucleic acid sequence in SEQ ID NO:12, or a variant nucleic acid molecule that hybridises to SEQ ID NO: 12 and encodes a polypeptide that has NAD reducing hydrogenase beta activity.

104. A method according to claim 98, wherein the nucleic acid molecule consists of a nucleotide sequence that encodes each polypeptide of SEQ ID NO: 3, 5, 8, 10 and 13.

105. A reaction vessel containing a host cell according to claim 81 and medium sufficient to support the growth of said cell.

106. A reaction vessel according to claim 105, wherein said vessel is a bioreactor.

107. A reaction vessel according to claim 105, wherein said vessel is a fermentor.

108. A method for producing hydrogen comprising:
i) providing a vessel comprising a host cell according to claim 81;
ii) providing cell culture conditions which facilitate hydrogen production by a cell culture contained in the vessel; and optionally iii) collecting hydrogen from the vessel.

109. An apparatus for the production and collection of hydrogen by a cell comprising:
i) a reaction vessel containing a host cell according claim 81; and
ii) a second vessel in fluid connection with said cell culture vessel wherein said second vessel is adapted for the collection and/or storage of hydrogen produced by cells contained in the cell culture vessel in (i).

110. The use of a cyanobacterial hydrogenase in a recombinant expression system for the production of hydrogen.

111. Use according to claim 110 wherein the cyanobacterial hydrogenase is encoded by a nucleic acid molecule selected from the group consisting of:
i) a nucleic acid molecule comprising the nucleotide sequence of SEQ ID NO: 1;
ii) a nucleic acid molecule having at least 70% identity to the nucleotide sequence of SEQ ID NO: 1 and which encodes a polypeptide that has hydrogenase activity;
iii) a nucleic acid molecule which hybridizes to the nucleic acid sequence of SEQ ID NO:1 and which encodes a polypeptide that has hydrogenase activity; or
iv) a nucleic acid molecule comprising a nucleotide sequence that is degenerate as a result of the genetic code to the sequences of i), ii) and iii) above.

112. A nucleic acid molecule represented by the nucleic acid sequence of SEQ ID NO:1.
